(19) United States
(12) Patent Application Publication
(10) Pub. No.: US 2011/0098241 A1
(43) Pub. Date: Apr. 28, 2011

(54) RAPAMYCIN ANALOGS AS ANTI-CANCER AGENTS

(75) Inventors: Connie L. Sun, Palo Alto, CA (US); Xiaoyuan Li, Los Altos, CA (US)

(73) Assignee: PONIARD PHARMACEUTICALS, INC.

(21) Appl. No.: 12/936,500

(22) PCT Filed: Apr. 13, 2009
(86) PCT No.: PCT/US09/02290

§ 371 Date: Dec. 22, 2010

Related U.S. Application Data
(60) Provisional application No. 61/044,849, filed on Apr. 14, 2008.

(51) Int. Cl. A61K 31/706; C07D 491/06; C07H 5/06; A61K 31/5377; A61K 31/4433; A61K 31/496; A61K 31/695; C12N 9/99; A61P 35/00

(52) U.S. Cl. 514/43; 540/456; 536/29.11; 514/233.2; 514/291; 514/253.03; 514/63; 435/184

(57) ABSTRACT

Analogs and derivatives of rapamycin are provided, wherein the analogs and derivatives can bind to FK-506 binding protein (FKBP), or inhibit the mTOR function of an FKBP, or both. The analogs and derivatives are rapamycin include the rapamycin skeleton substituted at the 42-hydroxyl group with certain specified chemically feasible groups. Methods of using the rapamycin analogs and derivatives in treatment of malconditions such as cancer, and methods of synthesizing the rapamycin analogs and derivatives, are provided.

### CROSS-REFERENCE TO RELATED APPLICATIONS

This application claims the priority of U.S. Ser. No. 61/044,849, filed Apr. 14, 2008, the disclosure of which is incorporated by reference herein in its entirety.

### BACKGROUND

Rapamycin, also known as sirolimus, is a macrocyclic organic compound including an ester, i.e., a macrolide, originally isolated from Streptomyces hygroscopicus, and is known to have immunosuppressant and antiproliferative properties. The mode of action of sirolimus is believed to be binding of the protein FKBP 12. The sirolimus-FKBP 12 complex is believed to inhibit the mTOR function, a serine/threonine protein kinase activity, through directly binding the mTOR Complex1 (mTORC1).

### SUMMARY

The present invention is directed to analogs and derivatives of the macrocyclic immunosuppressant rapamycin, methods of preparation of these analogs and derivatives, and their use in the treatment of malconditions including various types of cancer.

Various embodiments of the invention provide a compound of formula (I):

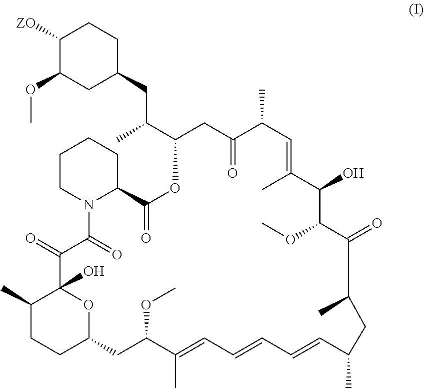

wherein Z comprises

(a) —C(O)NHS(O)₂N(R¹)(R²), wherein R¹ and R² are each independently H, alkyl, hydroxyalkyl, aminoalkyl, cycloalkyl, heterocyclyl, aryl, or heteroaryl, wherein any heterocyclyl independently comprises 1-3 heteroatoms comprising O, S, S(O), S(O)₂, or N; or, R¹ and R² together with a nitrogen atom to which they are bonded form a heterocycle ring which contains 0-3 additional heteroatoms comprising O, S, S(O), S(O)₂, or N, wherein any alkyl, hydroxyalkyl, aminoalkyl, aryl, heteroaryl, heterocyclyl, or heterocycle ring formed by R¹ and R² together with a nitrogen atom is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof;

R³ independently at each occurrence is H, alkyl, cycloalkyl, heterocyclyl, aryl, or heteroaryl, wherein any R³ except hydrogen is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and

R⁴ and R⁵ independently at each occurrence is H, alkyl, hydroxyalkyl, aminoalkyl, cycloalkyl, heterocyclyl, aryl, or heteroaryl; or R⁴ and R⁵ together with a nitrogen atom to which they are bonded comprises a saturated 5-, 6-, or 7-membered heterocyclic ring optionally comprising 0-3 additional heteroatoms comprising N, S S(O), S(O)₂ or O; wherein any alkyl, hydroxyalkyl, aminoalkyl, cycloalkyl, heterocyclyl, aryl, or heteroaryl, or heterocyclic ring formed by R⁴ and R⁵ together with a nitrogen atom, is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof;

or

(b) A-W—CH₂—(CH₂—O)ₘ—(CH(R))ₙ—, wherein

R is independently at each occurrence H or OR³;

m and n are each independently 0 to about 4;

W is a bond, C(O), C(O)C(O), S(O), S(O)₂, P(O)OR³, or P(O)NR⁴R⁵;

A comprises a saturated or unsaturated 5-, 6-, or 7-membered heterocyclyl containing one or more of N, O, S, S(O) or S(O)₂, wherein A is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and wherein A is bonded in any chemically feasible manner to CH₂;

R³ independently at each occurrence is H, alkyl, cycloalkyl, heterocyclyl, aryl, or heteroaryl, wherein any R³ except hydrogen is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and

R⁴ and R⁵ independently at each occurrence is H, alkyl, hydroxyalkyl, aminoalkyl, cycloalkyl, heterocyclyl, aryl, or heteroaryl; or R₄ and R₅ together with a nitrogen atom to which they are bonded comprises a saturated 5-, 6- or 7-membered heterocyclic ring comprising one or more of heteroatoms comprising N, S S(O), S(O)₂ or O; wherein any alkyl, hydroxyalkyl, aminoalkyl, alkylaminoalkyl, or aryl, or heterocyclic ring comprising R⁴ and R⁵ together, is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof;

or

(c) X—(CH₂)ₘYC(═O)(CH₂)ₙ—, wherein

m and n are each independently 0 to about 2;

Y is NR¹⁴, O, S, or a bond;

X comprises OR¹¹, or NR¹⁴R¹⁵, wherein

R¹¹ is H, alkyl, cycloalkyl, heterocyclyl, aryl, or heteroaryl, wherein any R¹¹ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;

R¹³ independently at each occurrence is H, alkyl, cycloalkyl, heterocyclyl, aryl, or heteroaryl, wherein any R¹³ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;

R¹⁴ and R¹⁵ independently at each occurrence is H, alkyl, hydroxyalkyl, aminoalkyl, cycloalkyl, heterocyclyl, aryl, or heteroaryl; or R¹⁴ and R¹⁵ together with a nitrogen atom to which they are bonded comprises a saturated 5-, 6- or 7-membered heterocyclic ring optionally comprising one or more additional heteroatoms comprising N, S S(O), S(O)₂ or O; wherein any alkyl, hydroxyalkyl, aminoalkyl, alkylaminoalkyl, or aryl, or heterocyclic ring comprising R¹⁴ and R¹⁵ together with a nitrogen atom is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;

or

(d) —X-A¹-(CH₂)ₙ—Y¹—Si(R²²)(R²³)(R²⁴)

wherein X comprises ((CHR²¹)ₘ;

R²¹ is independently at each occurrence H, alkyl, hydroxyl, alkoxyl, or amino;

m and n are independently 0 to about 3;

Y¹ is a bond, O(CH₂)ᵣ, NR¹⁴(CH₂)ᵣ, or S(CH₂)ᵣ, wherein r is 0 to about 3;

A¹ is a bond, O, NR¹⁴, S, cycloalkyl, or heterocyclyl;

R²², R²³ and R²⁴ are each independently alkyl, hydroxyalkyl, aminoalkyl, alkoxy, cycloalkyl, heterocyclyl, aryl, or heteroaryl;

wherein any alkyl, cycloalkyl, heterocyclyl, alkoxy, aryl, or heteroaryl is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;

R¹³ independently at each occurrence is H, alkyl, cycloalkyl, heterocyclyl, aryl, or heteroaryl, wherein any R¹³ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;

R¹⁴ and R¹⁵ independently at each occurrence is H, alkyl, hydroxyalkyl, aminoalkyl, cycloalkyl, heterocyclyl, aryl, or heteroaryl; or R¹⁴ and R¹⁵ together with a nitrogen atom to which they are bonded comprises a saturated 5-, 6- or 7-membered heterocyclic ring optionally comprising one or more additional heteroatoms comprising N, S S(O), S(O)₂ or O; wherein any alkyl, hydroxyalkyl, aminoalkyl, alkylaminoalkyl, or aryl, or heterocyclic ring comprising R¹⁴ and R¹⁵ together with a nitrogen atom is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof.

In various embodiments, pharmaceutical compositions comprising an effective amount of a compound of the invention and a pharmaceutically acceptable excipient are provided.

In various embodiments, pharmaceutical combinations comprising an effective amount of a compound of the invention and an effective amount of a second medicament are provided.

In various embodiments, pharmaceutical compositions comprising an effective amount of a compound of the invention, an effective amount of a second medicament, and a suitable excipient are provided.

In various embodiments, the invention provides a method of inhibiting the mTOR function of FKBP comprising contacting FKBP and an effective amount of the compound of the invention.

In various embodiments, the invention provides a method of treating a malcondition wherein binding of a ligand to FKBP, or inhibition of the mTOR function of FKBP, or both, is medically indicated, comprising administering the compound, composition, or combination of the invention to the patient in a dose, at a frequency of administration and for a duration of time sufficient to provide a beneficial effect to the patient.

### DETAILED DESCRIPTION

The term “treatment” is defined as the management and care of a patient for the purpose of combating the disease, condition, or disorder, for example one of the many types of conditions collectively referred to as “cancer”, and includes administering a compound of the present invention to prevent the onset of the symptoms or complications, or alleviating the symptoms or complications, or eliminating the disease, condition, or disorder.

As the term is used herein, “cancer” refers to solid tumors, hematopoietic malignancies, neoplasms, hyperplasias, malignant growths, and the like.

“FKBP” as the term is used herein refers to an FK506 Binding Protein. One bioactivity of FKBP is the “mTOR¹¹, or “mammalian target of rapamycin”, function, a serine/threonine protein kinase activity, as the term is used herein. Rapamycin is known to bind to FKBP and to inhibit this enzymatic activity, which is believed to be responsible at least in part for rapamycin's immunosuppressant and antiproliferative bioactivities.

“Treating” within the context of the instant invention means an alleviation of symptoms associated with a disorder or disease, or inhibition of further progression or worsening of those symptoms, or prevention or prophylaxis of the disease or disorder. Thus, treating a type of cancer includes slowing, halting or reversing the growth of the neoplasm and/or the control, alleviation or prevention of symptoms of the infection. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result by inhibition of FKBP mTOR activity. A therapeutically effective amount is also one in which any toxic or detrimental effects of compounds of the invention are outweighed by the therapeutically beneficial effects. For example, in the context of treating malconditions such as neoplasms or hyperproliferative diseases, a therapeutically effective amount of a FKBP mTOR inhibitor of the invention is an amount sufficient to exert a beneficial effect on the malcondition.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim.

When a substituent is specified to be an atom or atoms of specified identity, “or a bond”, a configuration is referred to when the substituent is “a bond” that the groups that are immediately adjacent to the specified substituent are directly connected to each other by a chemically feasible bonding configuration.

All chiral, diastereomeric, racemic forms of a structure are intended, unless a particular stereochemistry or isomeric form is specifically indicated. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like;
alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

The term “hydroxyl protecting group” or “O-protected” as used herein refers to those groups intended to protect an OH group against undesirable reactions during synthetic procedures and which can later be removed to reveal the amine. Commonly used hydroxyl protecting groups are disclosed in Protective Groups in Organic Synthesis, Greene, T. W.; Wuts, P. G. M., John Wiley & Sons, New York, N.Y., (3rd Edition, 1999). Hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. It is well within the skill of the ordinary artisan to select and use the appropriate hydroxyl protecting group for the synthetic task at hand.

In general, “substituted” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboyxlate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR′, OC(O)N(R′)₂, CN, CF₃, OCF₃, R′, O, S, C(O), S(O), methylenedioxy, ethylenedioxy, N(R′)₂, SR', SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R)C(O)N(R)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted.

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups can be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N−1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

Aryl groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to 20 ring members, whereas other such groups have 3 to 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. The phrase “heterocyclyl group” or “heterocycle” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

Heterocyclylalkyl groups are alkyl groups as defined above in which a hydrogen or carbon bond of an alkyl group is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group, including a cycloalkyl group, as are defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like.

“Halo” as the term is used herein includes fluoro, chloro, bromo, and iodo. A “haloalkyl” group includes mono-halo alkyl groups, and poly-halo alkyl groups wherein all halo atoms can be the same or different. Examples of haloalkyl include trifluoromethyl, 1,1-dichloroethyl, 1,2-dichloroethyl, 1,3-dibromo-3,3-difluoropropyl and the like.

The terms “aryloxy” and “arylalkoxy” refer to, respectively, an aryl group bonded to an oxygen atom and an aralkyl group bonded to the oxygen atom at the alkyl moeity. Examples include but are not limited to phenoxy, naphthyloxy, and benzyloxy.

An “acyl” group as the term is used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “amine” includes primary, secondary, and tertiary amines having, e.g., the formula N(group)₃ wherein each group can independently be H or non-H, such as alkyl, aryl, and the like. Amines include but are not limited to R—NH₂, alkylamines, arylamines, alkylarylamines, R₂NH wherein each R is independently selected, such as dialkylamines, diarylamines, aralkylamines, heterocyclylamines and the like, and R3N wherein each R is independently selected, such as trialkylamines, dialkylarylamines, alkyldiarylamines, triarylamines, and the like. An “amino” group is a substituent of the form —NH₂, —NHR, —NR₂, —NR₃⁺, wherein each R is independently selected, and protonated forms of each. The term “amine” also includes ammonium ions as used herein.

An “ammonium” ion includes the unsubstituted ammonium ion NH₄⁺, but unless otherwise specified, it also includes any protonated or quaternarized forms of amines. Thus, trimethylammonium hydrochloride and tetramethylammonium chloride are both ammonium ions, and amines, within the meaning herein.

The term “amide” (or “amido”) includes C- and N-amide groups, i.e., —C(O)NR₂, and —NRC(O)R groups, respectively. Amide groups therefore include but are not limited to carbamoyl groups (—C(O)NH₂) and formamide groups (—NHC(O)H).

The term “urethane” (or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

In various embodiments, the compound or set of compounds, such as are used in the inventive methods, can be any one of any of the combinations and/or sub-combinations of the above-listed embodiments.

Various embodiments of the invention include compounds of formula (I):

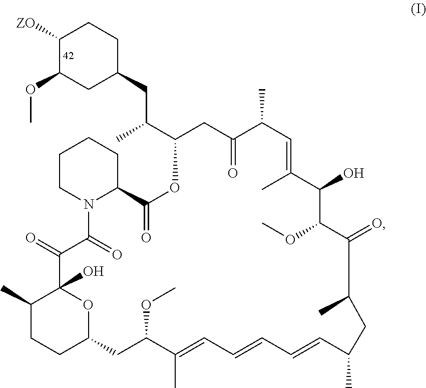

wherein a derivative is formed by bonding of a chemical group to the carbon-42 hydroxyl group, that is, the carbon atom bearing OZ in the above structure as indicated by the number “42”.

For example, Z can be a group of the formula —C(O)NHS(O)₂N(R¹)(R²), wherein R¹ and R² are each independently H, alkyl, hydroxyalkyl, aminoalkyl, cycloalkyl, heterocyclyl, aryl, or heteroaryl, wherein any heterocyclyl independently comprises 1-3 heteroatoms comprising O, S, S(O), S(O)₂, or N; or, R¹ and R² together with a nitrogen atom to which they are bonded form a heterocycle ring which contains 0-3 additional heteroatoms comprising O, S, S(O), S(O)₂, or N, wherein any alkyl, hydroxyalkyl, aminoalkyl, aryl, heteroaryl, heterocyclyl, or heterocycle ring formed by R¹ and R² together with a nitrogen atom is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof;

R³ independently at each occurrence is H, alkyl, cycloalkyl, heterocyclyl, aryl, or heteroaryl, wherein any R³ except hydrogen is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and

R⁴ and R⁵ independently at each occurrence is H, alkyl, hydroxyalkyl, aminoalkyl, cycloalkyl, heterocyclyl, aryl, or heteroaryl; or R⁴ and R⁵ together with a nitrogen atom to which they are bonded comprises a saturated 5-, 6-, or 7-membered heterocyclic ring optionally comprising 0-3 additional heteroatoms comprising N, S S(O), S(O)₂ or O; wherein any alkyl, hydroxyalkyl, aminoalkyl, cycloalkyl, heterocyclyl, aryl, or heteroaryl, or heterocyclic ring formed by R⁴ and R⁵ together with a nitrogen atom, is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof.

Compounds of this set can be viewed as substituted sulfamylacyl derivatives of rapamycin, bonded to the carbon-42 hydroxyl group of the rapamycin ring system wherein the terminal amino group of the sulfamate group can bear two independent substituents or can be incorporated into a heterocyclic ring. These substituents can be independently selected.

For example, in certain embodiments, one of R¹ or R² is hydrogen, and the other substituent is a monovalent group, such as alkyl, such as methyl. It is understood that the two structures are identicial when either of R¹ or R² is hydrogen and the other is a specific group. The alkyl group can itself be substituted, for example with one or two hydroxyl groups. More specifically, the group can be a hydroxyethyl group or a 2,3-dihydroxypropyl group. Or, the alkyl group can be substituted with a carboxyl group, for example R¹ or R² can be a carboxymethyl group.

In other embodiments, when one of R¹ or R² is hydrogen, the other can be an aryl group. The aryl group can itself be substituted, for example with a hydroxyl group. Thus, a p-hydroxyphenyl group is a specific example.

In still other embodiments, the other of R¹ or R² that is not hydrogen can be a heterocyclyl group, such as a hexahydropyranyl group. That group can itself bear substituents. When a hexahydropyranyl group bears hydroxyl and hydroxymethyl groups in the proper substitution pattern, the group is a pyranose form of a monosaccharide. More specifically, the group can by a glycosyl or a galactosyl group, bonded to the nitrogen atom via any carbon. When the heterocyclyl group is a tetrahydrofuranyl group, it can be substituted with hydroxyl and hydroxymethyl groups to provide a furanose form of a monosaccharide.

In other embodiment, both R¹ and R² are alkyl, such as substituted alkyl. For example, both R¹ and R² can be hydroxyethyl groups, respectively.

In yet other embodiments, R¹ and R² can together with the nitrogen atom to which they are bonded form a heterocyclic ring. For example the heterocyclic ring can be a pyrrolidine ring. The pyrrolidine ring can itself be substituted, such as with a hydroxyl group, in any chemically feasible position, such as the 3-position. It is understood that when substitution of a position can yield more than a single stereomeric form, all possible stereomeric forms are included.

In another set of embodiments according to the invention, Z can be A-W—CH₂—(CH₂—O)ₘ—(CH(R))ₙ—, wherein

R is independently at each occurrence H or OR³;

m and n are each independently 0 to about 4;

W is a bond, C(O), C(O)C(O), S(O), S(O)₂, P(O)OR³, or P(O)NR⁴R⁵;

A comprises a saturated or unsaturated 5-, 6-, or 7-membered heterocyclyl containing one or more of N, O, S, S(O) or S(O)₂, wherein A is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and wherein A is bonded in any chemically feasible manner to CH₂;

R³ independently at each occurrence is H, alkyl, cycloalkyl, heterocyclyl, aryl, or heteroaryl, wherein any R³ except hydrogen is optionally independently mono- or pluri-substituted with alkyl, hydroxyalkyl, aminoalkyl, halogen, oxo, OR², NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and

R⁴ and R⁵ independently at each occurrence is H, alkyl, hydroxyalkyl, aminoalkyl, cycloalkyl, heterocyclyl, aryl, or heteroaryl; or R₄ and R₅ together with a nitrogen atom to which they are bonded comprises a saturated 5-, 6- or 7-membered heterocyclic ring comprising one or more of heteroatoms comprising N, S S(O), S(O)₂ or O; wherein any alkyl, hydroxyalkyl, aminoalkyl, alkylaminoalkyl, or aryl, or heterocyclic ring comprising R⁴ and R⁵ together, is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof;

In various embodiments, m can be 0, such that the O-42 substituent is a carbon chain bearing the A heterocyclyl group. For example, the carbon chain can be an ethyl chain, when n=1 and R is all H, or a propyl chain, when n=2 and R is all H. In other embodiments the chain can include oxygen atoms, in polyethylene glycol (PEG) repeating units.

In various embodiments A can be a piperidinyl ring bonded directly to the CH₂—(CH₂—O)ₘ, —(CH(R))ₙ, when W is a bond. The heterocyclyl, e.g., piperidinyl, ring can be bonded to the chain, which can be the carbon chain or the PEG chain. The piperidinyl ring can be unsubstituted, or it can be substituted, for example with a alkoxycarbonyl group, ROC(O)—. More specifically, R can be a methyl group, providing a CH₃OC(O)— substituent on the piperidinyl ring. The piperidinyl ring can be attached to the carbon chain or oxycarbon chain in any chemically feasible manner; for example the piperidinyl ring can be attached by any carbon atom, for example by carbon number 4, or by the nitrogen atom of the ring. Substituents can be disposed on the ring in any chemically feasible manner.

In other embodiments, the heterocyclic ring A can be bonded to the carbon or oxycarbon chain —CH₂—(CH₂—O)ₘ—(CH(R))ₙ—, via W in any chemically feasible manner when W is a bond, C(O), C(O)C(O), S(O), S(O)₂, P(O)OR³, or P(O)NR⁴R⁵. The heterocyclic ring can be attached via the W group to the carbon or oxycarbon chain —CH₂—(CH₂—O)ₘ—(CH(R))ₙ by any chemically feasible configuration for bonding to the terminal CH₂ group; for example the heterocyclic ring can be connected by one of the ring carbon atoms, or by a ring nitrogen atom. For example, the heterocyclic ring can be bonded via a ring nitrogen atom to the carbon or oxycarbon chain via a carbonyl (C(O)), oxalyl (C(O)C(O)), sulfenyl (S(O), or sulfonyl (S(O)₂) group. More specifically, if the heterocyclic ring is a morpholinyl ring, it can be bonded to the carbon or oxycarbon chain via the ring nitrogen atom through a sulfonyl group to the terminal CH₂ group of the carbon or oxycarbon chain, thus forming a sulfonamide group. Or, a ring nitrogen can be connected to the terminal CH₂ group of the carbon or oxycarbon chain via a carbonyl group, thus forming an amide group.

In various other embodiments of the invention, Z can be X—(CH₂)ₘYC(═O)(CH₂)ₙ—, wherein

m and n are each independently 0 to about 2;

Y is NR¹⁴, O, S, or a bond;

X comprises OR¹¹, or NR¹⁴R¹⁵, wherein

R¹¹ is H, alkyl, cycloalkyl, heterocyclyl, aryl, or heteroaryl, wherein any R¹¹ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;

R¹³ independently at each occurrence is H, alkyl, cycloalkyl, heterocyclyl, aryl, or heteroaryl, wherein any R¹³ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³,S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;

R¹⁴ and R¹⁵ independently at each occurrence is H, alkyl, hydroxyalkyl, aminoalkyl, cycloalkyl, heterocyclyl, aryl, or heteroaryl; or R¹⁴ and R¹⁵ together with a nitrogen atom to which they are bonded comprises a saturated 5-, 6- or 7-membered heterocyclic ring optionally comprising one or more additional heteroatoms comprising N, S S(O), S(O)₂ or O; wherein any alkyl, hydroxyalkyl, aminoalkyl, alkylaminoalkyl, or aryl, or heterocyclic ring comprising R¹⁴ and R¹⁵ together with a nitrogen atom is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof.

In various embodiments of the invention, m and n can both be 0. In various embodiments, Y can be absent. When both these conditions are met, Z is a carbonate ester when X is OR¹¹, or a carbmate when X is NR¹⁴R¹⁵, wherein X is bonded directly to the carbonyl group which is in turn directly bonded to the hydroxyl group disposed on carbon-42 of the rapamycin ring scaffold. For example, NR¹⁴R¹⁵ can be an unsubstituted or a substituted heterocyclic ring, such as a piperidinyl, or piperazinyl, or a morpholinyl ring, bonded via the nitrogen atom to the carbonyl group. More specifically, in an embodiment, NR¹⁴R¹⁵ can be a piperazinyl ring bonded via the 1-nitrogen atom to the carbonyl group, wherein the 4-nitrogen atom bears an alkyl group, such as a methyl group. In other embodiments, NR¹⁴R¹⁵ can be a piperidinyl or a morpholinyl ring bonded by respective nitrogen atoms to the carbonyl group. In specific examples, all these rings can be otherwise unsubstituted.

In other embodiments, m and n can each independently be 1 or 2. For example, in various embodiments, m can be 1 or 2 and Y can be absent, such that the carbonyl group is bonded directly to a 1 or 2 carbon unit, which is in turn bonded to X, thus forming an aminoalkyl or an alkoxyalkyl group bonded to the carbonyl. The carbonyl can in turn either be bonded directly to the rapamycin 42-hydroxyl group, forming an ester bond, or a 1 or 2 carbon chain can be interspersed, such that the carbonyl is a ketone carbonyl, and the rapamycin 42-hydroxyl forms an ether linkage with the 1 or 2 carbon chain.

In yet other embodiments, an atom Y can be disposed between the carbonyl and the (CH₂)ₙ, group, wherein Y can be an optionally substituted nitrogen atom, an oxygen atom, or a sulfur atom, thus providing an amide, ester, or thioester linkage respectively. In various embodiments, Y is a NH group, which can be linked via a 1 or 2 carbon atom linker to X. For example, the Z group can be of the structures (heterocyclyl)-CH₂CH₂NHC(O)CH₂— or (heterocyclyl)CH₂CH₂NHC(O)—, bonded to the rapamycin 42-hydroxyl.

In various embodiments, Z can be —X-A¹-(CH₂)ₙ—Y¹—Si(R²²)(R²³)(R²⁴)

wherein X comprises ((CHR²¹)ₘ;

R²¹ is independently at each occurrence H, alkyl, hydroxyl, alkoxyl, or amino;

m and n are independently 0 to about 3;

Y¹ is a bond, O(CH₂)ᵣ, NR¹⁴(CH₂)^{r} or S(CH₂)ᵣ, wherein r is 0 to about 3;

A¹ is a bond, O, NR¹⁴, S, cycloalkyl, or heterocyclyl;

R²², R²³ and R²⁴ are each independently alkyl, hydroxyalkyl, aminoalkyl, alkoxy, cycloalkyl, heterocyclyl, aryl, or heteroaryl;

wherein any alkyl, cycloalkyl, heterocyclyl, alkoxy, aryl, or heteroaryl is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;

R¹³ independently at each occurrence is H, alkyl, cycloalkyl, heterocyclyl, aryl, or heteroaryl, wherein any R¹³ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;

R¹⁴ and R¹⁵ independently at each occurrence is H, alkyl, hydroxyalkyl, aminoalkyl, cycloalkyl, heterocyclyl, aryl, or heteroaryl; or R¹⁴ and R¹⁵ together with a nitrogen atom to which they are bonded comprises a saturated 5-, 6- or 7-membered heterocyclic ring optionally comprising one or more additional heteroatoms comprising N, S S(O), S(O)₂ or O; wherein any alkyl, hydroxyalkyl, aminoalkyl, alkylaminoalkyl, or aryl, or heterocyclic ring comprising R¹⁴ and R¹⁵ together with a nitrogen atom is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof.

For example, in various embodiments, m can be 0 and A¹ can be a bond, such that the (CH₂)ₙ group is bonded directly to the rapamycin 42-hydroxyl group. In other embodiments, Y¹ is a bond. More specifically, when m is 0, A¹ is a bond, and Y¹ is a bond, the compound of formula (I) comprises a silyl ether or a silylalkyl ether of the rapamycin 42-hydroxyl group, depending on the value of n. When n is 1 and R²², R²³ and R²⁴ are each a methyl group, the compound of formula (I) includes a O(42)-trimethylsilylmethyl ether of rapamycin. In other embodiments, one or more of R²², R²³ and R²⁴ can each independently be a substituted alkyl group, for example, a hydroxyethyl group. For example, a compound of formula (I) can include a dimethyl-2-hydroxyethylsilylmethyl ether at the 42-hydroxyl group. In another embodiment, a polyethyleneglycol chain segment is disposed between the silylmethyl ether and the 42-hydroxyl group; for example as in the structure —CH₂CH₂OCH₂CH₂OCH₂Si(R²²)(R²³)(R²⁴).

In other embodiments, Y¹ can be an oxygen atom, such that the compound of formula (I) includes a siloxyalkyl ether of the rapamycin 42-hydroxyl group.

Compounds of the invention are believed to act by binding to FKBP or inhibition of the mTOR function of FKBP, or both. The bioactivity of the inventive compounds can be evaluated using bioassay procedures known in the art, as are described below. All exemplary compounds shown below were found to have IC₅₀ values of less than about 5 μM, many less than about 1 μM.

In various embodiments, the invention provides a method of preparation of a compound of the invention, comprising

contacting a compound of formula (II):

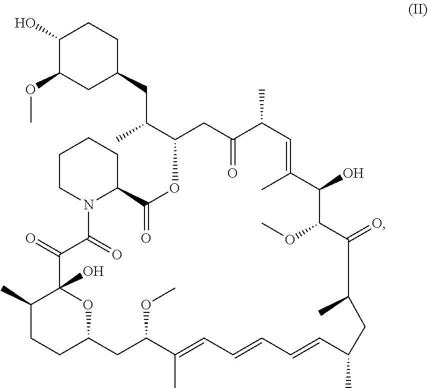

with a hydroxyl-protecting group reagent, to provide a compound of formula (III):

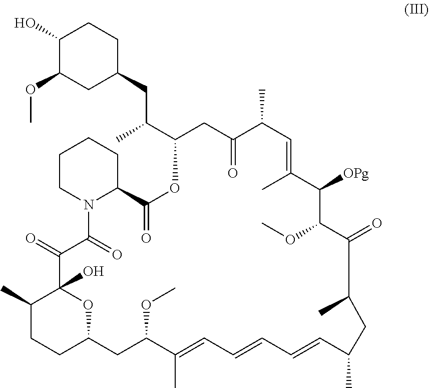

wherein Pg is a hydroxyl protecting group, then

contacting the compound of formula (III) with sulfurisocyanatidic chloride to provide a compound of formula (IV):

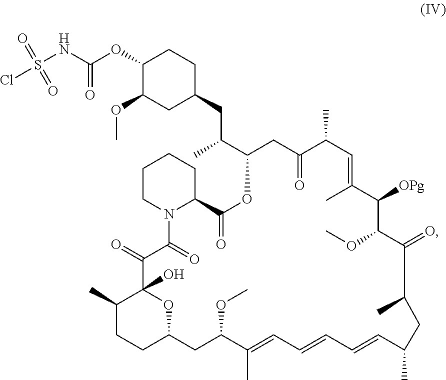

then, contacting the compound of formula (IV) with NH(R¹)(R²) to provide the compound of the invention. The hydroxyl protecting group can be a silyl ether, such as a tert-butyl-dimethylsilyl ether.

In various embodiments, the invention provides a method of preparing a compound of the invention comprising contacting a compound of formula (II):

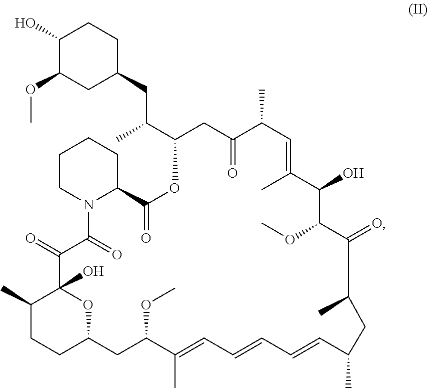

and a compound of formula Z-Lg, wherein Lg is a leaving group, to provide the compound of the invention. For example, the compound of formula Z—X can be a compound of formula Z—O-Tf, wherein Tf signifies a triflate ester. The hydroxyl protecting group can be a silyl ether, such as a tert-butyl-dimethylsilyl ether.

In various embodiments, the invention provides a method of preparing a compound of the invention comprising contacting a compound of formula (II):

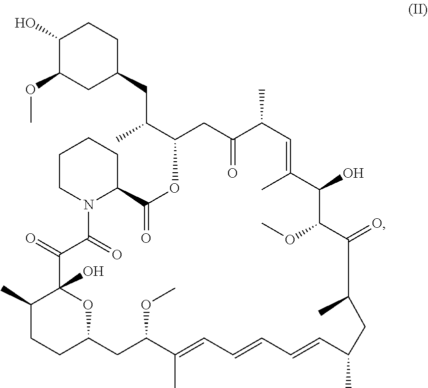

with a hydroxyl-protecting group reagent, to provide a compound of formula (III):

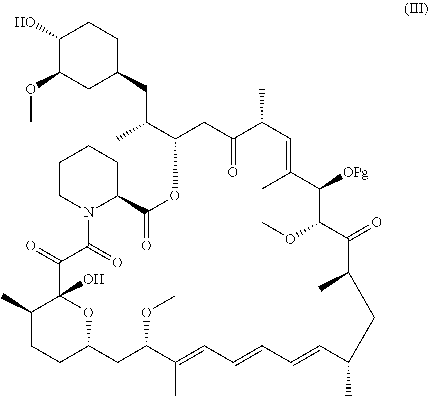

wherein Pg is a hydroxyl protecting group, then

contacting the compound of formula (III) with a reagent of formula Z-phenyl-O—C(═O)-Lg, wherein Z is one or more electron withdrawing groups disposed on phenyl and Lg is a leaving group, to provide a compound of formula (VI):

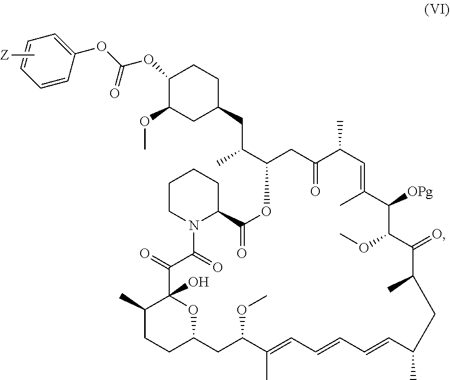

wherein Z signifies the one or more electron withdrawing groups; then, contacting the compound of formula (VI) with NH(R¹²)(R¹³) to provide the compound of the invention. For example, the reagent of formula Z-phenyl-O—C(═O)-Lg can be a mononitrophenoxycarbonyl chloride or a dinitrophenoxycarbonyl chloride. The hydroxyl protecting group can be a silyl ether, such as a tert-butyl-dimethylsilyl ether.

In various embodiments, the invention provides a method of preparing a compound of the invention, comprising contacting a compound of formula (II):

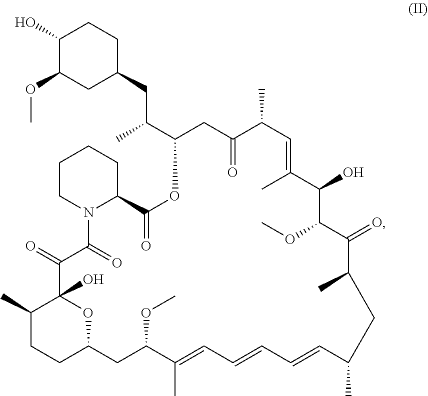

with a hydroxyl-protecting group reagent, to provide a compound of formula (III):

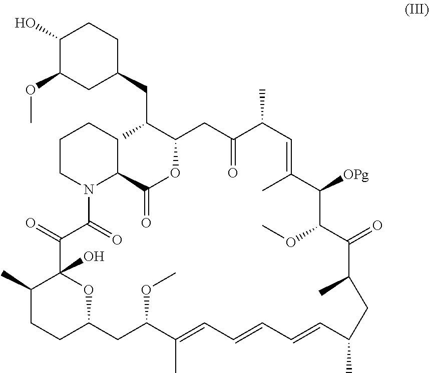

wherein Pg is a hydroxyl protecting group, then

contacting the compound of formula (III) with an activated haloacetate to provide a compound of formula (VII):

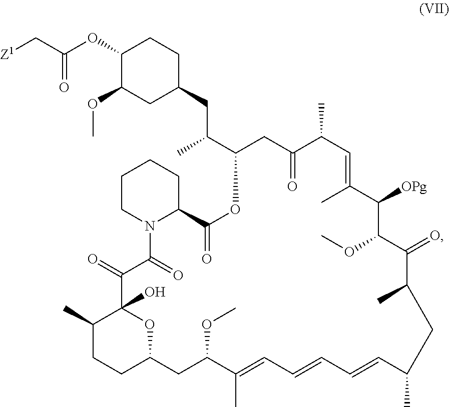

wherein Z¹ is a halogen;

then, contacting the compound of formula (VII) with NH(R¹²)(R¹³) to provide the compound of the invention. For example, Z′ can be bromo. The hydroxyl protecting group can be a silyl ether, such as a tert-butyl-dimethylsilyl ether.

In various embodiments, the invention provides a method of preparing a compound of the invention, comprising contacting a compound of formula (II):

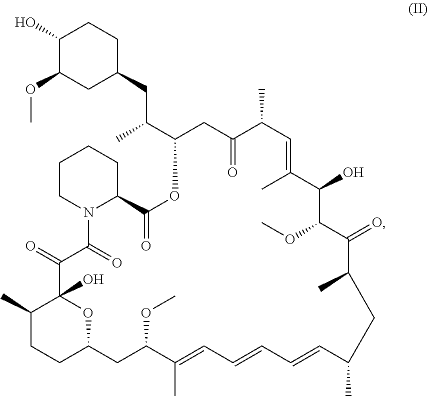

and a compound of formula Lg-A¹-(CH₂)ⁿ—Si(R²²)(R²³)(R²⁴), wherein Lg is a leaving group, to provide the compound of claim 20. For example, Lg can be —O—SO₂CF₃. The hydroxyl protecting group can be a silyl ether, such as a tert-butyl-dimethylsilyl ether.

### EXAMPLES

The following abbreviations are used throughout the Examples:
DCM dichloromethane
DMF N,N-dimethylformamide
Et₃N triethylamine
EtOAc ethyl acetate
g grams
h hours
min minutes
mL milliliters
mmole millimoles
THF tetrahydrofuran
TMS trimethylsilyl
TMS—Cl trimethylsilylchloride
° C. degrees Celsius
˜range (e.g. 5˜10° C.)

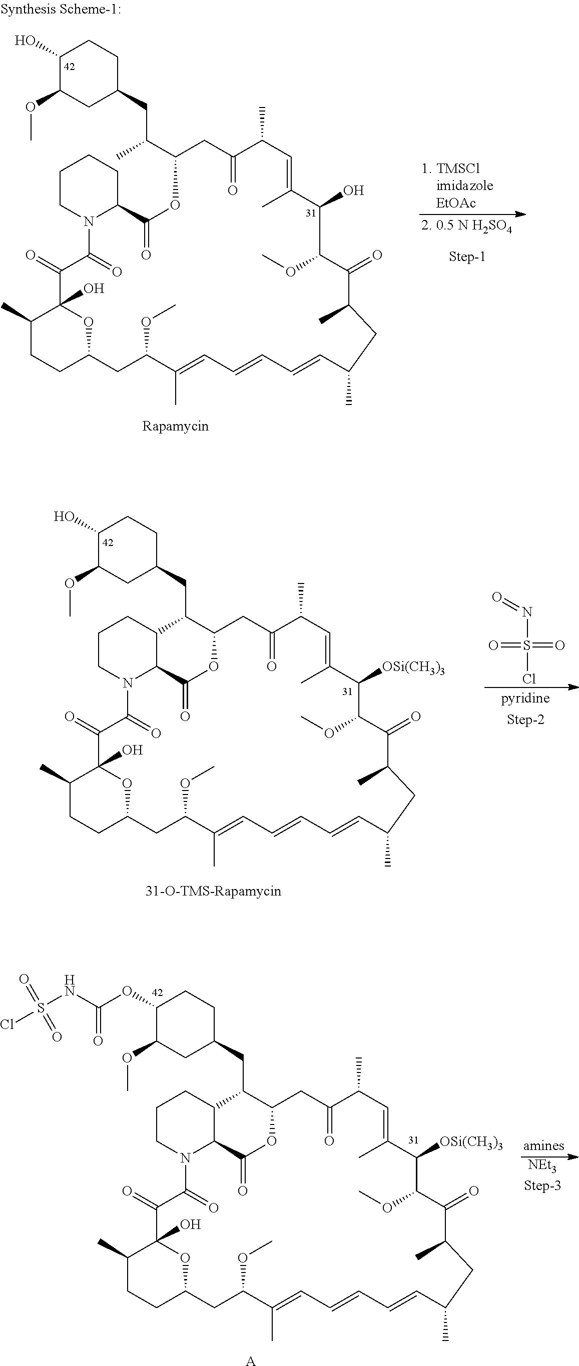

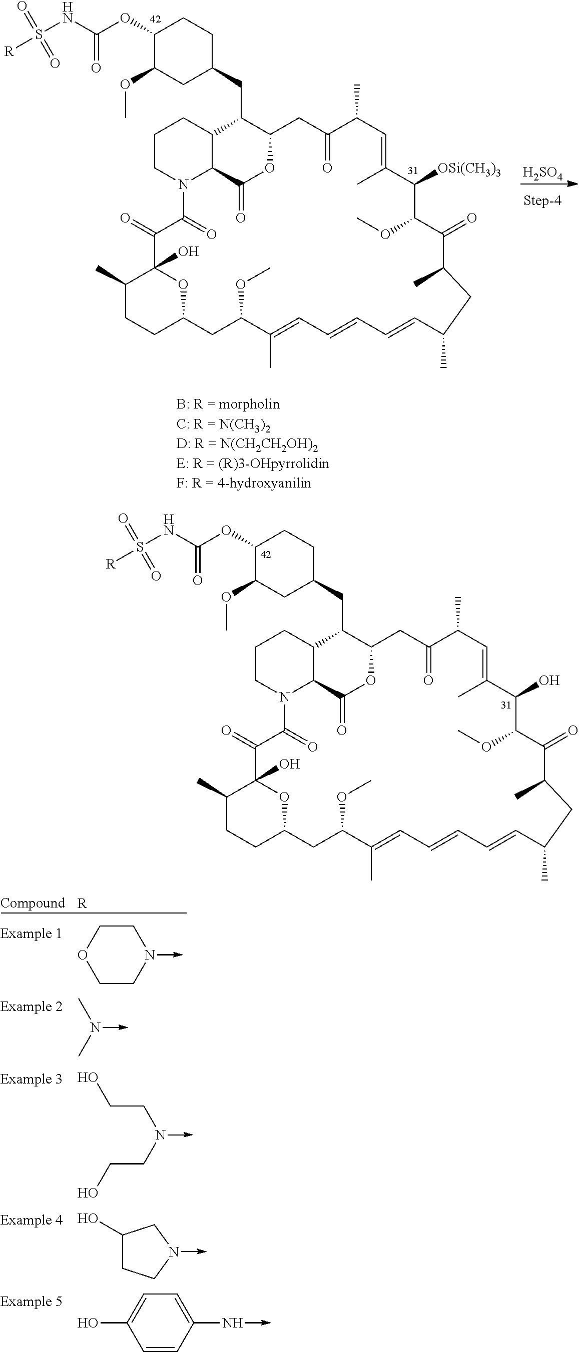

### Example 1

### 42-O-[Morpholinosulfonylcarbamyl]-rapamycin

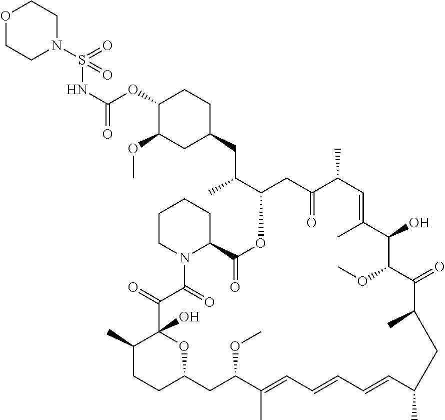

### Step-1: 31-O—TMS-rapamycin

To an ice-cooled solution of rapamycin (5.5 g, 6 mmol) and imidazole (3.2 g, 48 mmol) in ethyl acetate (30 mL) was added chlorotrimethylsilane (5.2 g, 48 mmol) dropwise by syringe. The reaction mixture was stirred at room temperature for 30 minutes. Upon completion of the reaction, sulfuric acid (0.5 N, 24 mL) was added dropwise. The reaction mixture was stirred at 0° C. for 1.5 h, diluted with brine, and extracted with ethyl acetate (3×30 mL). The organic layer was dried over anhydrous sodium sulfate and concentrated. The residue was purified by silica gel chromatography eluted with ethyl acetate-petroleum ether (1:1) to obtain 31-O-TMS-rapamycin (4.8 g, 81%).

### Step-2 and Step-3: 42-O-Morpholinosulfonylcarbamate-31-O-TMS-rapamycin (B)

To a solution of 31-O-TMS-rapamycin (1.7 g, 1.7 mmol) and pyridine (0.4 g, 5.1 mmol) in CH₂Cl₂ (20 mL) at 0° C. under nitrogen was added sulfurisocyanatidic chloride (ClSO₂(NO), 0.25 g, 1.7 mmol) dropwise by syringe. The reaction mixture was stirred at 0° C. for 1 h before morpholine (0.4 g, 5.1 mmol) was added. The mixture was stirred for another 2 h, diluted with brine, and extracted with ethyl acetate (3×15 mL). The organic layer was dried over anhydrous sodium sulfate and concentrated. The residue was purified by silica gel chromatography eluted with ethyl acetate-petroleum ether (1:1.5) to obtain B as a white solid (25%).

### Step-4: 42-O-Morpholinosulfonylcarbamyl-rapamycin

To the solution of intermediate B (510 mg, 0.43 mmol) in acetone (20 mL) at 0° C. was added sulfuric acid (0.5 N, 1.7 mL). The mixture was stirred for 1.5 h at 0° C., diluted with brine, and extracted with ethyl acetate (10×10 mL). The organic layer was dried over anhydrous sodium sulfate and concentrated. The residue was purified by silica gel chromatography eluted with EtOAc-petroleum ether (1:1.5) to obtain the title compound (0.42 g, 88%).

¹H NMR (400 MHz, CDCl₃) δ 3.75-3.77 (m, 4H), 3.40-3.41 (m, 4H), 4.76 (s, 1H, OH), 5.41 (d, J=9.2 Hz, 1H, OH), 5.46-5.57 (m, 1H, vinyl-H), 6.11-6.42 (m, 3H, vinyl-H).

m/z (relative intensity, %): 1105 [M−1]^{+.} (100)

### Example 2

### 42-O-[Dimethylaminosulfonylcarbamyl]-rapamycin

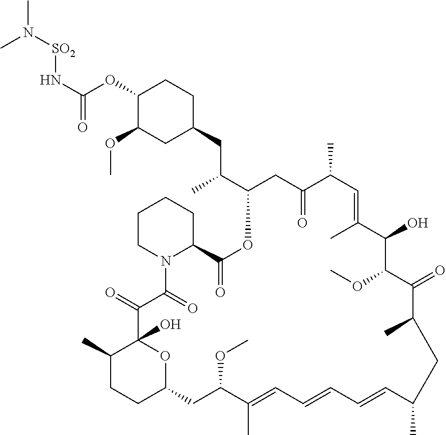

To a solution of 31-O-TMS-rapamycin (2.5 g, 2.5 mmol) and pyridine (0.7 g, 10 mmol) in anhydrous CH₂Cl₂ (20 mL) at 0° C. under nitrogen was added sulfurisocyanatidic chloride (1.09 g, 7.5 mmol) dropwise by syringe. The reaction mixture was stirred at 0° C. for 0.5 h. To this reaction mixture was added Et₃N (0.67 g) followed by dimethylamine hydrochloride (1.61 g, 7.5 mmol). The reaction mixture was stirred for another 1.5 h at 0° C., diluted with brine, and extracted with ethyl acetate (10×30 mL). The organic layer was dried over anhydrous sodium sulfate and concentrated. The residue was purified by silica gel chromatography eluted with EtOAc-petroleum ether (1:1.5) to obtain C as a white solid (25%).

To the solution of C (600 mg, 0.5 mmol) in acetone (20 mL) was cooled to 0° C. and added sulfuric acid (0.5 N, 1.8 mL). The mixture was stirred for 1.5 h at 0° C., diluted with brine, and extracted with ethyl acetate (10×30 mL). The organic layer was dried over anhydrous sodium sulfate and concentrated. The residue was purified by silica gel chromatography eluted with EtOAc-petroleum ether (1:1.5) to obtain the title compound (0.42 g, 91%).

¹H NMR (400 MHz, CDCl₃) δ 2.97 (s, 6H, N(CH₃)₂), 4.76 (s, 1H, OH), 5.41 (d, J=9.2 Hz, 1H, OH), 5.46-5.57 (m, 1H, vinyl-H), 6.11-6.42 (m, 3H, vinyl-H).

m/z (relative intensity, %): 1063 [M−1]^{+.} (100)

### Example 3

### 42-O—[N,N-Bis(2-hydroxyethyl)aminosulfonylcarbamyl]-rapamycin

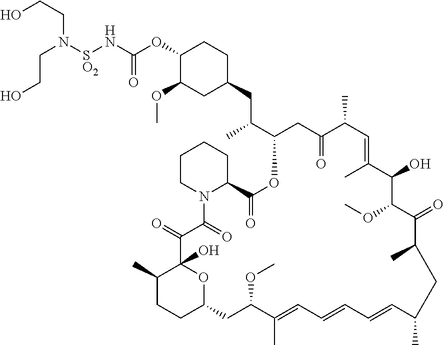

To a solution of 31-O-TMS-rapamycin (2.5 g, 2.5 mmol) and pyridine (0.7 g, 10 mmol) in anhydrous CH₂Cl₂ (20 mL) at 0° C. under nitrogen was added sulfurisocyanatidic chloride (1.09 g, 7.5 mmol) dropwise by syringe. The reaction mixture was stirred at 0° C. for 0.5 h. To this reaction mixture was added Et₃N (0.67 g) followed by diethanolamine (0.52 g, 5 mmol). The reaction mixture was stirred for another 1.5 h at 0° C., diluted with brine, and extracted with ethyl acetate (10×30 mL). The organic layer was dried over anhydrous sodium sulfate and concentrated. The residue was purified by silica gel chromatography eluted with EtOAc-methanol (40:1) to obtain D as a white solid (21%)

To the solution of D (240 mg, 0.2 mmol) in acetone (20 mL) was added sulfuric acid (0.5 N, 0.8 mL) at 0° C. The reaction mixture was stirred for 1.5 h, diluted with brine, and extracted with EtOAc (10 mL×3). The organic layer was dried over anhydrous Na₂SO₄ and concentrated. The residue was purified by silica gel chromatography eluted with CHCl₃-CH₃OH (40:1) to obtain the title compound (0.16 g, 82%).

¹H NMR (400 MHz, CDCl₃) δ 3.51-3.58 (m, 4H, 2CH₂), 3.85-3.90 (m, 4H, 2CH₂), 4.60-4.64 (m, 1H), 4.67 (s, 1H), 5.51 (d, J=10 Hz, 1H), 5.49-5.55 (m 1H, vinyl-H), 5.98 (d, J=10.0 Hz, 1H, vinyl-H), 6.11-6.17 (m, 1H, vinyl-H), 6.27-6.42 (m, 2H, vinyl-H).

m/z (relative intensity, %): 1122 [M−1]^{+.} (100)

### Example 4

### 42-O—[(R)-3-hydroxypyrrolidin-1-ylsulfonylcarbamyl]-rapamyein

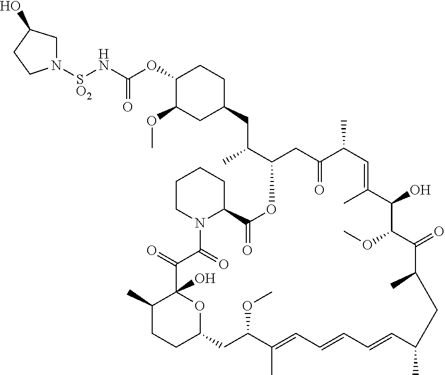

To a solution of rapamycin-31-O-TMS (2.5 g, 2.5 mmol) and pyridine (0.7 g, 10 mmol) in anhydrous THF (20 mL) at 0° C. under nitrogen was added sulfurisocyanatidic chloride (1.09 g, 7.5 mmol) dropwise by syringe. The reaction mixture was stirred at 0° C. for 0.5 h. To this reaction mixture was added Et₃N (0.67 g) followed by (R)-3-hydroxypyrrolidine hydrochloride (0.71 g, 6.1 mmol). The reaction mixture was stirred for another 1.5 h at 0° C., diluted with brine, and extracted with ethyl acetate (10×30 mL). The organic layer was dried over anhydrous sodium sulfate and concentrated. The residue was purified by silica gel chromatography eluted with EtOAc-methanol (40:1) to obtain E as a white solid (25%).

To the solution of E (200 mg, 0.17 mmol) in acetone (20 mL) was added sulfuric acid (0.5 N, 0.8 mL) at 0° C. The reaction mixture was stirred for 1.5 h, diluted with brine, and extracted with EtOAc (10 mL×3). The organic layer was dried over anhydrous Na₂SO₄ and concentrated. The residue was purified by silica gel chromatography eluted with CHCl₃-CH₃OH (40:1) to obtain the title compound (0.17 g, 77%).

¹H NMR (400 MHz, CDCl₃) δ 2.51-2.73 (m, 4H), 2.04-2.27 (m 6H), 3.63-3.73 (m, 7H), 4.45, (s, br, 1H), 4.18 (s, br, 1H), 4.62 (s, br, 1H), 5.42 (d, J=9.6 Hz, 1H), 5.43-5.53 (m 1H, vinyl-H), 5.98 (d, J=10.4 Hz, 1H, vinyl-H), 6.11-6.16 (m, 1H, vinyl-H), 6.29-6.37 (m, 2H, vinyl-H), 7.49 (s, 1H).

m/z (relative intensity, %): 1105 [1\4-1]^{+.} (100)

### Example 5

### 42-O-[4-Hydroxyanilinsulfonylcarbamyl]-rapamycin

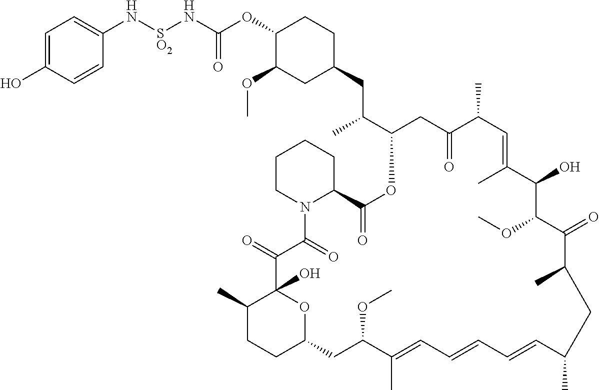

To a solution of 31-O-TMS-rapamycin (2.5 g, 2.5 mmol) and pyridine (0.7 g, 10 mmol) in anhydrous CH₂Cl₂ (20 mL) at 0° C. under nitrogen was added sulfurisocyanatidic chloride (1.09 g, 7.5 mmol) dropwise by syringe. The reaction mixture was stirred at 0° C. for 0.5 h. To this reaction mixture was added Et₃N (0.67 g) followed by 4-aminophenol (0.71 g, 6.6 mmol). The reaction mixture was stirred for another 1.5 h at 0° C., diluted with brine, and extracted with ethyl acetate (10×30 mL). The organic layer was dried over anhydrous sodium sulfate and concentrated. The residue was purified by silica gel chromatography eluted with EtOAc-methanol (40:1) to obtain F as white solid (26%).

To the solution of F (200 mg, 0.17 mmol) in acetone (20 mL) was added sulfuric acid (0.5 N, 0.8 mL) at 0° C. The reaction mixture was stirred for 1.5 h, diluted with brine, and extracted with EtOAc (10 mL×3). The organic layer was dried over anhydrous Na₂SO₄ and concentrated. The residue was purified by silica gel chromatography eluted with CHCl₃-CH₃OH (40:1) to obtain the title compound (0.14 g, 73%).

¹H NMR (400 MHz, CDCl₃) δ 5.10 (s, br, 1H), 5.24 (d, J=3.2 Hz, 1H), 5.36-5.49 (m, 2H), 5.99 (d, J=10.8 Hz, 1H, vinyl-H), 6.07-6.13 (m, 1H, vinyl-H), 6.22-6.39 (m, 2H, vinyl-H), 6.83 (d, J=8.0 Hz, 1H), 7.07 (d, J=8.0 Hz, 2H), 7.40 (s, br, 1H).

m/z (relative intensity, %): [M−1]^{+.} 1126 (100).

### Example 6

### 42-O-[4-Methylpiperazine-1-carboxy]-rapamycin

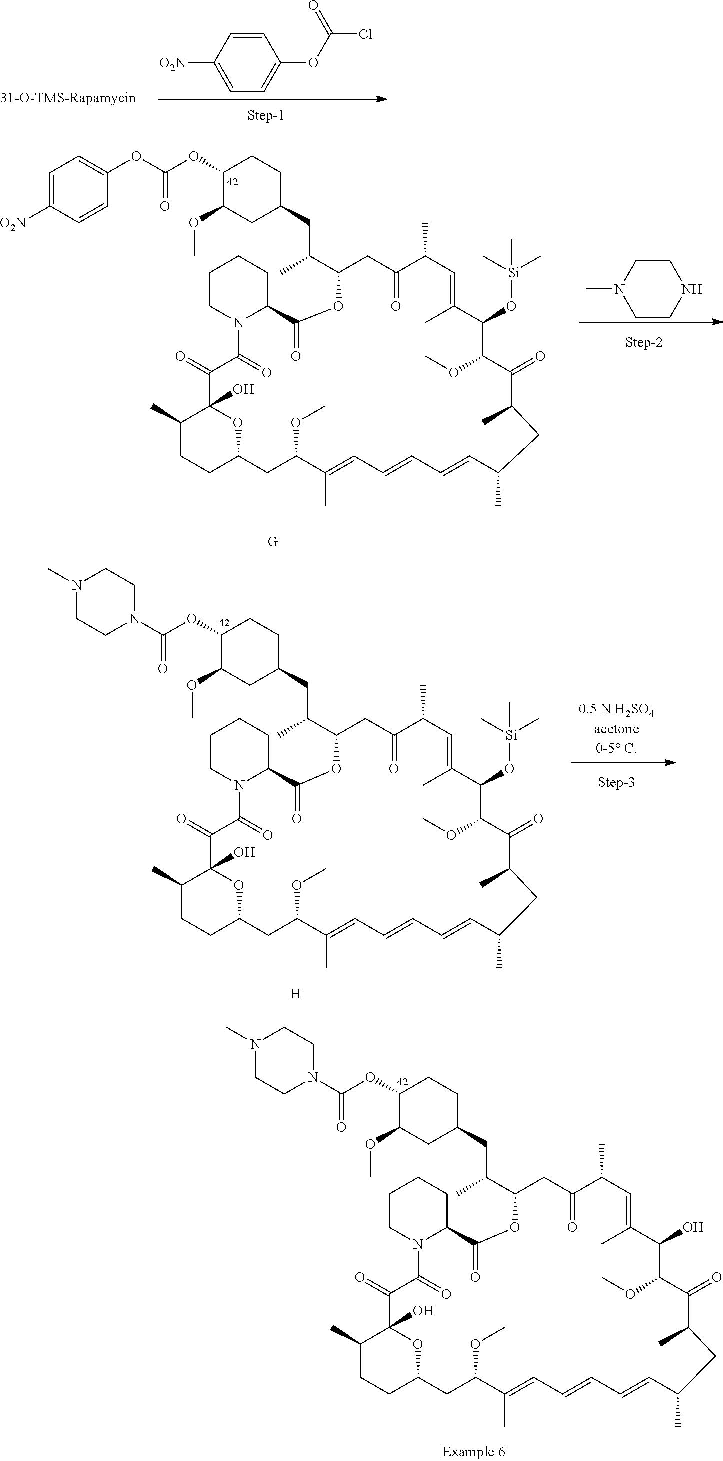

### Step-1: 42-O-(4-Nitrophenvoxycarboxy)-31-O-TMS-rapamycin (G)

To a solution of 31-O-TMS-rapamycin (5.1 g, 5.17 mmol) and pyridine (4.0 g, 51.7 mmol) in dichloromethane (30 mL) at −10° C. ˜−5° C. was added a solution of 4-nitrophenylchloroformate (1.5 g, 7.76 mmol) in dichloromethane (20 mL) dropwise. The reaction mixture stirred for 3 h with the temperature gradually increased from −10° C. to room temperature, quenched with water (300 mL), and extracted with dichloromethane (60 mL). The organic layer was washed with water (300 mL), dried over anhydrous sodium sulfate, and concentrated to afford G, which was used without further purification.

### Step-2: 42-O-(4-Methylpiperazine-1-carboxy)-31-O-TMS-rapamycin (H)

To the solution of compound G (5.8 g, 5.04 mmol) and triethylamine (1 g, 10.2 mmol) in DMF (50 mL) at 0° C. was added N-methylpiperazine slowly. The reaction mixture was stirred for 4 h with the temperature rising from 0° C. to room temperature, quenched with water (300 mL) and extracted with ethyl acetate (3×150 mL). The organic layer was washed with water (2×300 mL) and brine, dried over anhydrous sodium sulfate, and concentrated to give compound H as a light yellow solid (3.8 g, 67%) which was used without further purification.

### Step-3: 42-O-[4-Methylpiperazine-1-carboxy)-rapamycin

To the solution of H (200 mg, 0.18 mmol) in acetone (3 mL) and water (3 mL) at 0° C. was added sulfuric acid (0.5 N, 0.5 mL). The reaction mixture was stirred at 0° C. overnight, diluted with water (5 mL), neutralized with sodium bicarbonate, extracted with ethyl acetate (3×5 mL). The organic layer was washed with water and brine, dried over anhydrous sodium sulfate, and concentrated. The residue was purified by silica gel chromatography eluted with methanol-ethyl acetate (1:4) to afford the title compound as an off-white powder.

¹H NMR (400 MHz, CDCl₃) δ 3.1 (s, 3H, NCH₃), 5.41 (d, J=9.2 Hz, 1H, OH), 5.46-5.57 (m, 1H, vinyl-H), 6.11-6.42 (m, 3H, vinyl-H).

### Example 7

### 42-O—[(R)-3-Hydroxypyrrolidin-1-yl)acetyl]-rapamycin

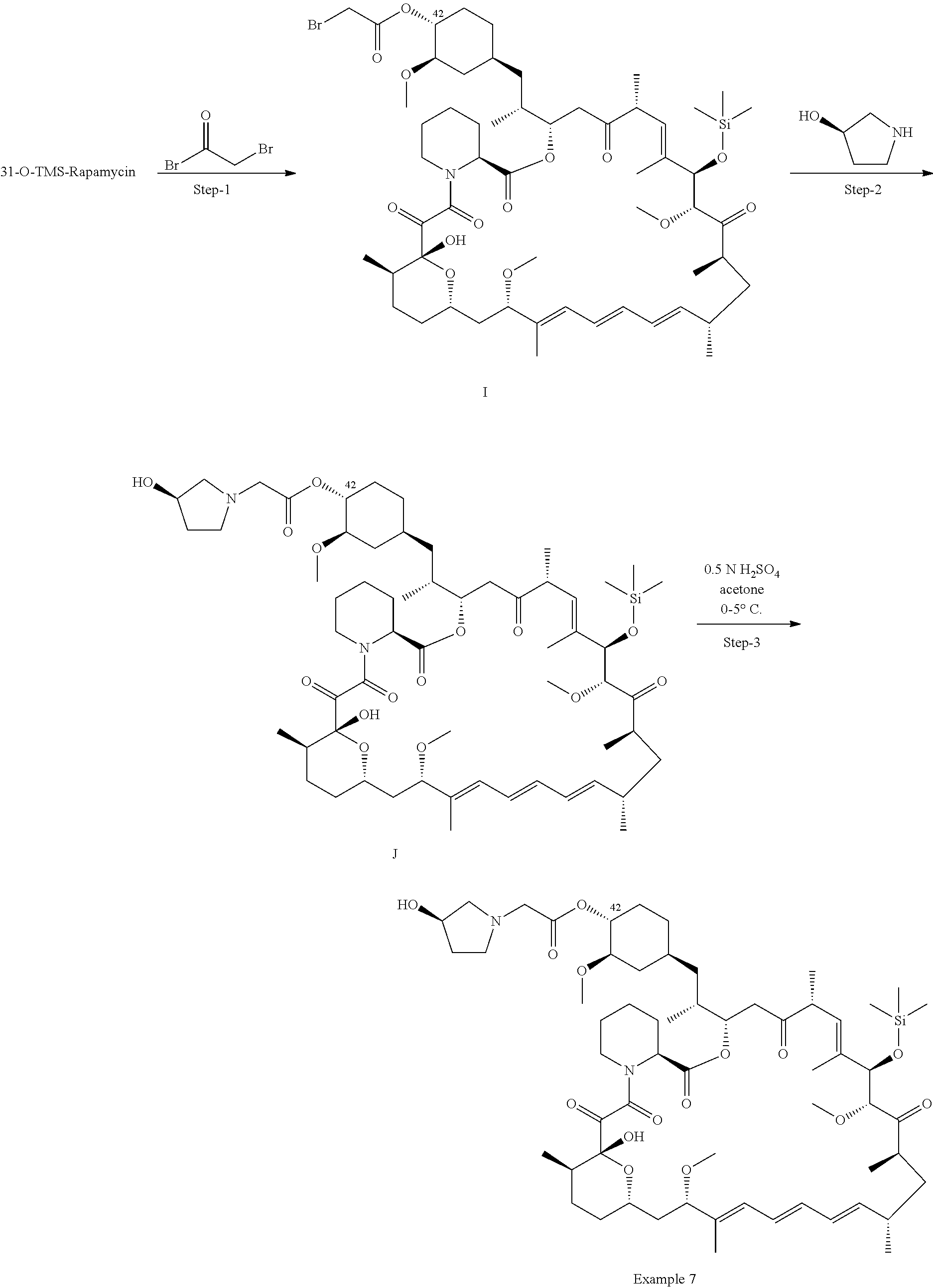

### Step-1: 42-O-(2-Bromoacetyl)-31-O-TMS-rapamycin (I)

To a solution of 31-O-TMS-rapamycin (0.2 g, 0.2 mmol) and triethylamine (0.2 g, 2 mmol) in dichloromethane (10 mL) was added 2-bromoacetyl bromide dropwise at 0° C. The reaction mixture was stirred for 6 h while the temperature rose to room temperature, concentrated, extracted with ethyl acetate. The organic layer was washed with brine, dried over anhydrous Na₂SO₄ and concentrated to give crude I which was used without further purification.

### Step-2: 42-O—[(R)-3-Hydroxypyrrolidin-1-yl)acetyl]-31-O-TMS-rapamycin

To the solution of I in DMF (10 mL) was added K₂CO₃ (50 mg, 0.4 mmol) and (R)-3-hydroxypyrrolidine hydrochloride salt (22 mg, 0.2 mmol). The reaction mixture was stirred at 40° C. for 6 h. Small quantities of (n-Bu₄N)₂SO₄ were added to the reaction mixture. The resultant mixture was stirred at 40° C. for another 8 h, cooled to room temperature, quenched with water, and extracted with ethyl acetate. The organic layer was washed with brine, dried over anhydrous Na₂SO₄, and concentrated to give crude J which was used without further purification.

### Step-3: 42-O—[(R)-3-Hydroxypyrrolidin-1-yl)acetyl]-rapamycin

To the solution of J (7.0 g, 7 mmol) in acetone (100 mL) was added 0.5 N H₂SO₄ (14 mL) dropwise at 0° C. The reaction mixture was stirred overnight while the reaction temperature was gradually warmed up to room temperature and concentrated. The red oil residue was purified on silica gel chromatography eluted with ethyl acetate-petroleum ether (1:1) to afford the title compound (2.0 g, 29%).

¹H NMR (300 MHz, CDCl₃) δ 4.08-4.18 (m, 4H), 2.0-2.1 (m, 8H), 5.99-6.40 (m, 3H, vinyl).

m/z (relative intensity, %): 985 [M−1]^{+.} (100)

### Example 8

### 42-O-[2-(4-Hydroxypiperidin-1-yl)acetyl]-rapamycin

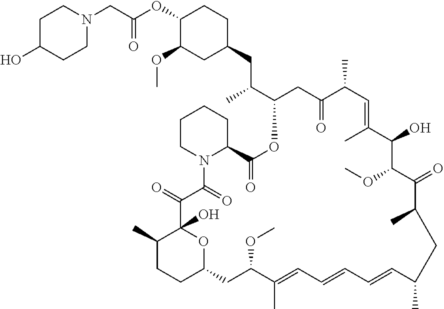

The title compound was prepared using the same procedure as Example 7, substituting 4-hydroxypiperidine hydrochloride for 4-hydroxypyrrolidine hydrochloride.

¹H NMR (400 MHz, CDCl₃) δ 3.88-4.00 (m, 4H), 5.28 (m, 2H), 5.46-5.57 (m, 1H, vinyl-H), 6.11-6.42 (m, 3H, vinyl-H).

### Example 9

### 42-O-[2-(Piperidin-4-yl)ethyl]-rapamycin

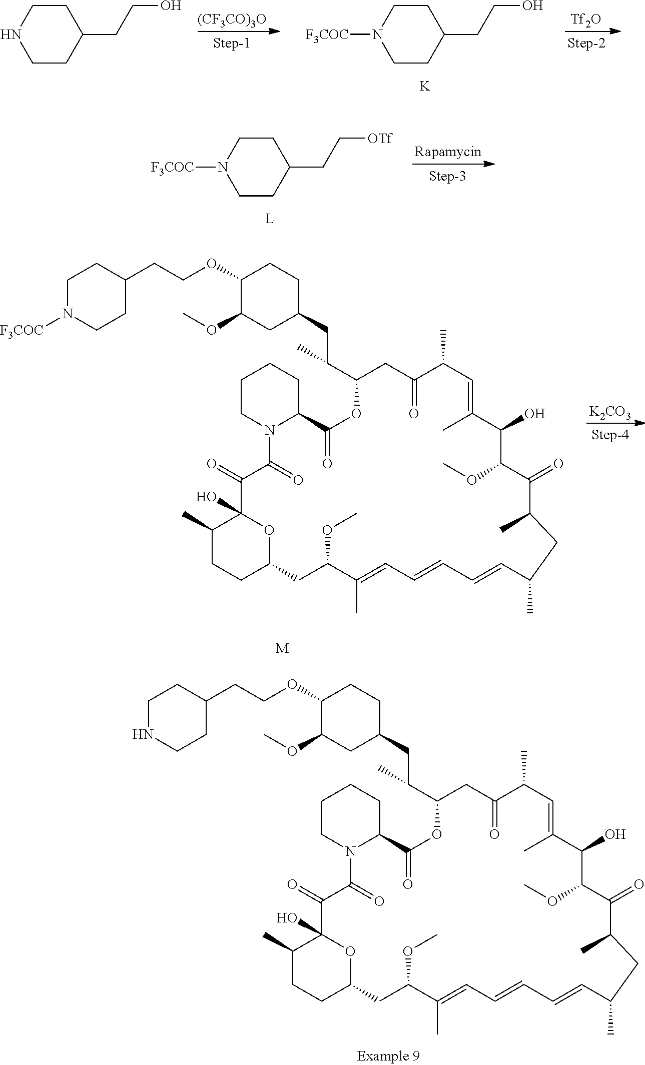

### Step-1: 2,2,2-Trifluoro-1-(4-(2-hydroxyethyl)piperidin-1-yl)ethanone (K)

To a mixture 2-(piperidin-4-yl)ethanol (2.58 g, 20 mmol) and triethylamine (4 mL, 30 mmol) in dichloromethane (50 mL) was added trifluoroacetic anhydride (2.8 mL, 20 mmol) dropwise at 0° C. The reaction mixture was stirred at room temperature for 2 h, and washed sequentially with hydrochloric acid (1 N), saturated NaHCO₃ and brine. The organic layer was dried over anhydrous Na₂SO₄, filtered, and concentrated. The residue was purified by silica gel chromatography eluted with hexane-ethyl acetate (5:1 to 1:1) to give 2,2,2-trifluoro-1-(4-(2-hydroxyethyl)piperidin-1-yl)ethanone as a colorless oil (2.1 g, 46%).

¹H NMR (400 MHz, DMSO-d₆) 1.05-1.10 (m, 2H), 1.36-1.39 (m, 2H), 1.70-1.81 (m, 3H), 2.82-2.88 (t, 1H), 3.17-3.24 (t, 1H), 3.42-3.46 (m, 2H), 3.81-3.84 (d, 1H), 4.24-4.28 (d, 2H), 4.41-4.43 (t, 1H).

m/z (relative intensity, %): 226 [M+1]⁺.

### Step-2: 2-(1-(2,2,2-Trifluoroacetyl)piperidin-4-yl)ethyl trifluoromethane sulfonate (L)

2,2,2-Trifluoro-1-(4-(2-hydroxyethyl)piperidin-1-yl)ethanone (K) (1.8 g, 8.0 mmol), and 2,6-lutidine (1.03 g, 9.6 mmol) were dissolved in DCM (50 mL). Triflic anhydride (Tf₂O, 2.7 g, 9.6 mmol) was added dropwise under N₂ at −5° C. The reaction mixture was stirred at room temperature for 2 h, and washed sequentially with hydrochloric acid (1 N), saturated NaHCO₃ and brine. The organic layer was dried over anhydrous Na₂SO₄, filtered, and concentrated. The residue was purified by silica gel chromatography eluted with hexane-ethyl acetate (2:1 to 1:1) to give 2-(1-(2,2,2-trifluoroacetyl)-piperidin-4-yl)ethyl trifluoromethanesulfonate as a light yellow oil (2.80 g, 98%).

¹HNMR (400 MHz, DMSO-d₆): 1.10-1.16 (m, 2H), 1.61-1.81 (m, 5H), 2.83-2.89 (t, 1H), 3.18-3.25 (t, 1H), 3.81-3.85 (d, 1H), 4.25-4.33 (m, 3H).

### Step-3: 42-O-[2,2,2-Trifluoro-1-(4-(2-hydroxyethyl)piperidin-1-yl)ethyl]rapamycin (M)

To a solution of rapamycin (500 mg, 0.55 mmol) and 2,6-lutidine (250 mg, 2.5 mmol) in toluene (5 mL) at room temperature under nitrogen was added 2-(1-(2,2,2-trifluoroacetyl)piperidin-4-yl)ethyl trifluoromethanesulfonate (0.78 g, 2.2 mmol). The reaction mixture was stirred at room temperature for 30 min, 0° C. for 2 h, then concentrated. The crude product was purified by silica gel chromatography eluted with hexane-ethyl acetate (1:1 to 1:2) to give the title compound as a light yellow solid (350 mg, 45%).

m/z (relative intensity, %): 1119 [M−1]^{+.}, 1143 [M+Na]^{+.}.

### Step-4. 42-O-[1-(4-(2-Hydroxyethyl)piperidin-1-yl)ethyl]rapamycin

A mixture of 42-O-[2,2,2-trifluoro-1-(4-(2-hydroxyethyl) piperidin-1-yl) ethyl] rapamycin (M) (50 mg, 0.045 mmol) and K₂CO₃ (16 mg, 0.12 mmol) in MeOH (1.5 mL) and H₂O (0.25 mL) was stirred at room temperature for 30 min and extracted with ethyl acetate. The organic layer was washed with NH₄Cl solution and brine, dried over MgSO₄, and concentrated. The crude product was purified by silica gel chromatography gave the title compound as a white solid (20 mg, 44%).

¹H NMR (400 MHz, CDCl₃) δ 1.0-2.02 (m, 2H).

m/z (relative intensity, %): 1026 [M+1]^{+.} (100).

### Example 10

### 42-O-[3-(4-Methoxycarbonyl-piperidin-1-yl)propyl]-rapamycin

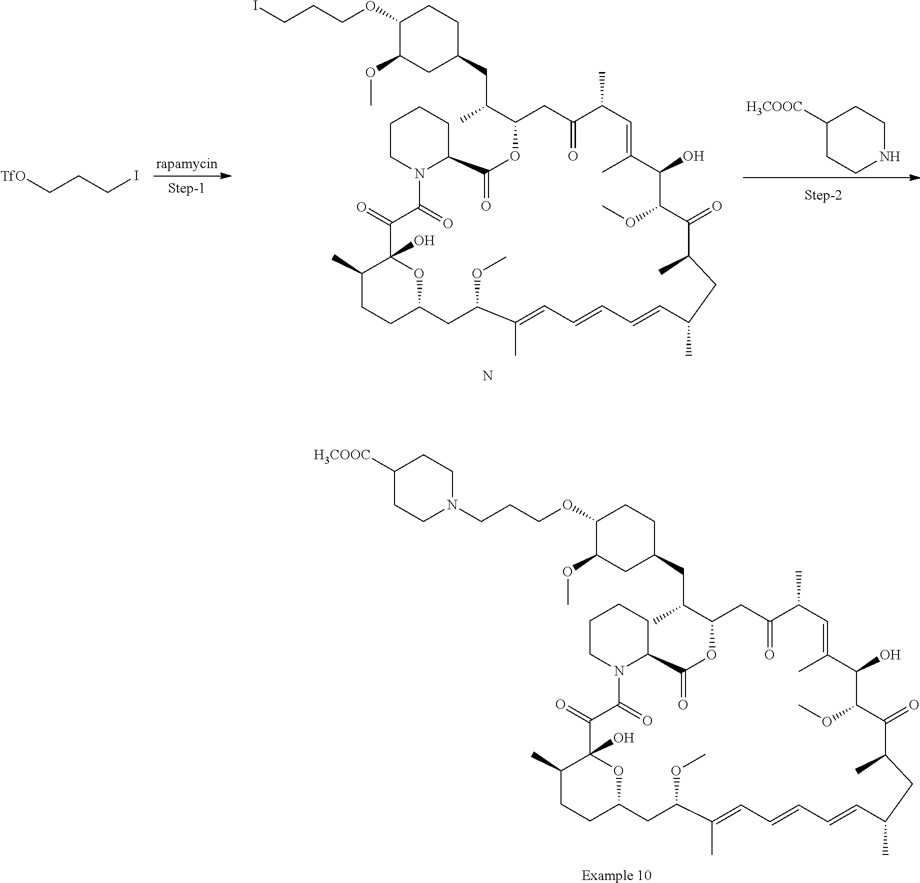

### Step-1: 42-O-[3-Iodopropyl]-rapamycin (N)

To a solution of rapamycin (0.5 g, 0.55 mmol) in dichloromethane (2 mL) was added Hunig's base (N,N-diisopropylethylamine, 4.93 mL, 28.4 mmol) and 3-iodopropyltrifluoromethanesulfonate (1.35 g, 4.36 mmol) sequentially. The reaction mixture was heated to 60° C., stirred for 1.5 h, and diluted with ethyl acetate (50 mL). The organic layer was washed with 1 N aqueous HCl (50 mL), water (50 mL), and brine (40 mL), dried over anhydrous sodium sulfate, and concentrated. The residue was purified by silica gel chromatography eluted with ethyl acetate-hexane (2:3) to afford the intermediate N (0.34 g, 58%).

### Step-2: 42-O-[3-(4-Methoxycarbonyl-piperidin-1-yl)propyl]-rapamycin

To the solution of N in dichloromethane (2 mL) was added Hunig's base (0.35 mL, 2.0 mmol) and methyl piperidine-4-carboxylate (100 mg, 0.1 mmol) sequentially. The reaction mixture was stirred at room temperature and diluted with ethyl acetate (50 mL). The organic layer was washed with water and brine, dried over anhydrous sodium sulfate, and concentrated. The residue was purified by silica gel chromatography eluted with ethyl acetate-methanol-triethylamine (8:1:1) to afford the title compound (98 mg, 97%).

m/z (relative intensity, %): 1096 [M−1]^{+.} (100)

### Example 11

### 42-O-[Trimethylsilyl-methyl]-rapamycin

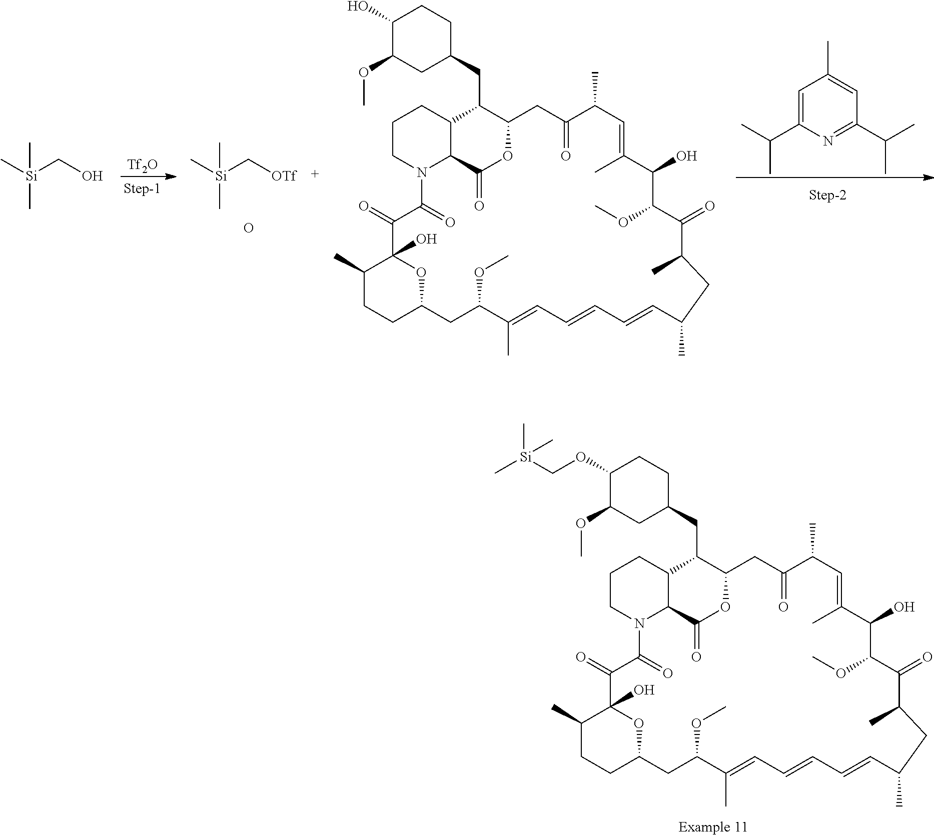

### Step-1: (Trimethylsilyl)methyl trifluoromethanesulfonate (O)

To a solution of (trimethylsilyl)methanol (4.26 g, 40.88 mmol) in dichloromethane (DCM) (80 ml) was added a solution of triflic anhydride (Tf₂O, 17.3 g, 61.32 mmol) in DCM (80 ml) at 0° C. The reaction mixture was stirred overnight while the temperature was maintained at 0° C. in a bath of ice-salt, washed four times with brine (80 ml), extracted one time with DCM (80 mL). The organic layers was dried over anhydrous MgSO₄ and concentrated. The residue was purified by distillation (fraction boiling at 80° C.) to give 0 (6.2 g, 64%) as a colorless liquid.

¹H NMR (400 MHz, CDCl₃) δ 0.18 (9H, s), 4.25 (2H, s).

### Step-2: 42-O-[Trimethylsilyl-methyl]-rapamycin

To a solution of rapamycin (800 mg, 0.88 mmol) was added 2,6-di-tert-butyl-4-methylpyridine (2.69 g, 13.1 mmol) in toluene (10 ml) and O (3.05 g, 12.9 mmol) sequentially at room temperature. The reaction mixture was warmed up, stifled at 60° C. for 2 h, and concentrated. The residue was purified by silica gel chromatography eluted with EtOAc-petroleum ether (1:1) to afford the title compound (67 mg, 7%) as a white solid. ¹H NMR (400 MHz, CDCl₃) δ 1.22-1.42 (m, 5H), 0.01 (s, 9H).

m/z (relative intensity, %): 1023 [M+Na]^{+.} (100)

### Example 12

### 42-O-[2-(Trimethylsilan-methoxy)-ethyl]-rapamycin

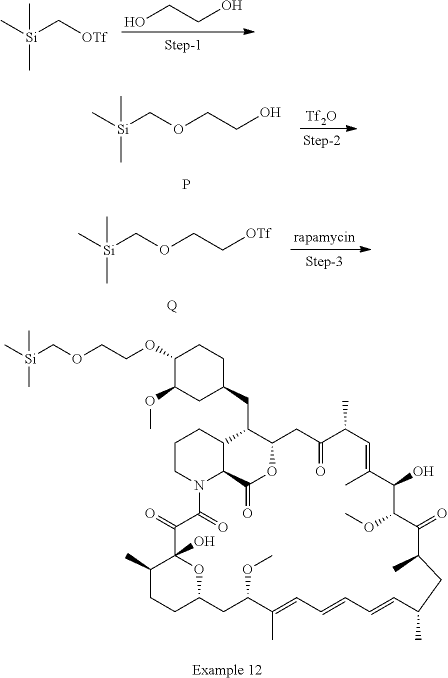

### Step-1: 2-[(Trimethylsilyl)methoxy]ethanol (P)

To ethane-1,2-diol (20 ml) was added (trimethylsilyl)methyl trifluoromethanesulfonate (800 mg, 3.39 mmol) dropwise. The resulting solution was stirred for 2 hours at room temperature, extracted three times with DCM (150 ml). The organic layers was washed with brine (4×50 mL), dried over anhydrous MgSO₄, and concentrated to give P (400 mg, 80%) as a colorless liquid which was used without further purification.

¹H NMR (300 MHz, CDCl₃) δ 3.64 (t, J=4.5 Hz, 2H), 3.47 (m, 2H), 3.11 (s, 2H), 0.05 (s, 9H)

### Step-2: 2-[(Trimethylsilyl)methoxy]ethyl trifluoromethanesulfonate (Q)

To a solution of Tf₂O (17 g, 60.3 mmol) in DCM (100 ml) was added a solution of P (6 g, 40.5 mmol) in DCM (10 ml) dropwise at 0° C. The resulting solution was stirred at room temperature overnight, quenched with ice water (100 mL). The organic layer was washed with brine (5×20 mL), and concentrated. The residue was purified by distillation under reduced pressure (20 mm Hg) and the fraction was collected at 60° C. to afford Q (9 g, 79%) as a colorless liquid.

¹H NMR (300 MHz, CDCl₃) δ: 4.59 (t, J=4.5 Hz, 2H), 3.70 (t, J=4.5 Hz, 2H), 3.16 (s, 2H), 0.05 (s, 9H).

### Step-3: 42-O-[2-(Trimethylsilylmethoxy)-ethyl]-rapamycin

To the solution of rapamycin (200 mg, 0.22 mmol) in 1,2-dichloroethane (3 ml) was added 2,6-diisopropyl-1-methylpiperidine (508 mg, 2.78 mmol) and Q (780 mg, 2.78 mmol) sequentially. The reaction mixture was warmed to 60° C., stirred for additional 2 h, and concentrated. The residue was purified by silica gel chromatography eluted with ethyl acetate-petroleum ether (1:1) to afford the title compound (58 mg, 24%) as a white solid.

¹H NMR (300 MHz, CDCl₃): δ 6.14-6.38 (m, 3H, vinyl), 4.81 (s, 1H, OH), 1.12-1.42 (m, 5H), 0.01 (s, 6H), 0.04 (s, 3H).

m/z (relative intensity, %): 1067 [M+Na]^{+.} (100%)

The following compounds were prepared using the same protocol for Example 12.

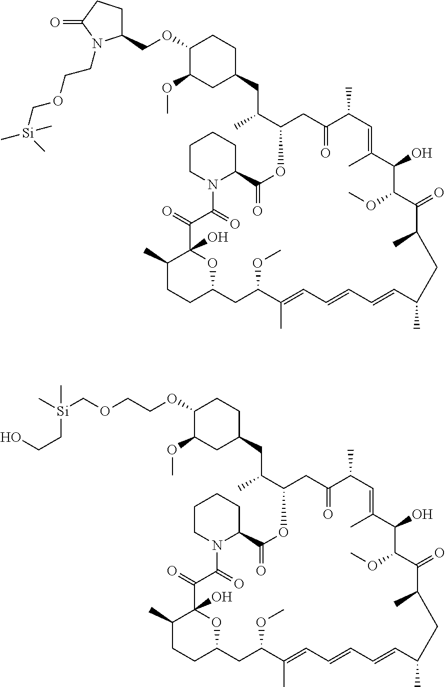

### Example 13

### 42-O-[2-(4-(2-Hydroxyethyl)piperidin-1-yl)acetyl]rapamycin

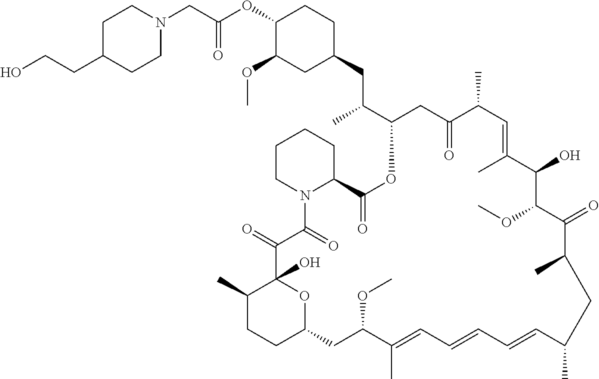

The title compound was prepared using the same procedure as Example 7, substituting 4-(hydroxyethyl)piperidine hydrochloride as reagent.

¹H NMR (400 MHz, DMSO-d₆) δ 5.75 (s, 1H, OH)

m/z (relative intensity, %): 1082 [M−1]^{+.} (75)

### Example 14

### 42-O-[2-(Bis(2-hydroxyethyl)amino)acetyl]-rapamycin

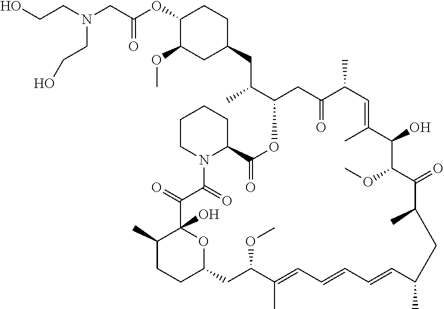

The title compound was prepared using the same procedure as Example 7, substituting bis(hydroxyethyl)amine as reagent.

¹H NMR (400 MHz, CDCl₃) δ 2.58-2.78 (m, 6H), 3.38-3.48 (m, 6H).

m/z (relative intensity, %): 1058 [M−1]^{+.} (78)

### Example 15

### 42-O-(2-Hydroxypiperidincarbonyl)-rapamycin

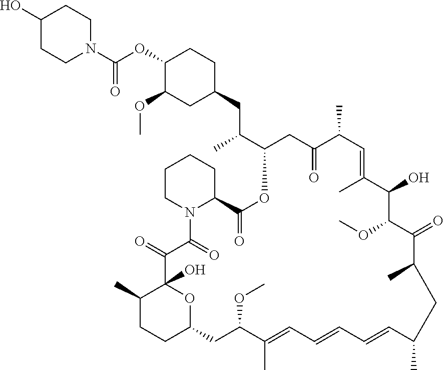

The title compound was prepared using the same procedure as Example 6, except substituting 4-hydroxypiperidine as the reagent.

¹H NMR (400 MHz, CDCl₃) δ 4.80 (s, 1H, OH), 4.30-4.55 (m, 4H).

m/z (relative intensity, %): 1039 [M−1]^{+.} (100)

### Example 16

### 42-O-(2-Morpholinoethylaminocarbonyl)-rapamycin

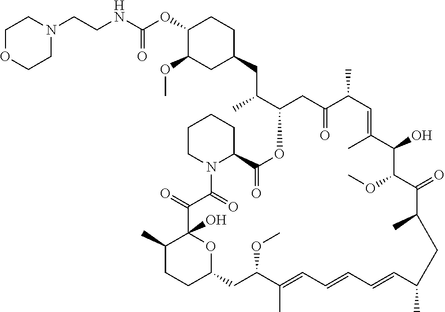

The title compound was prepared using the same procedure as Example 6, except substituting morpholinylethylamine as the reagent.

¹H NMR (400 MHz, CDCl₃) δ 2.45-2.49 (m, 6H), 3.66-3.74 (m, 6H).

m/z (relative intensity, %): 1069 [M−1]^{+.} (100)

### Example 17

### 42-O-[3-(Morpholinosulfonyl)propyl]-rapamycin

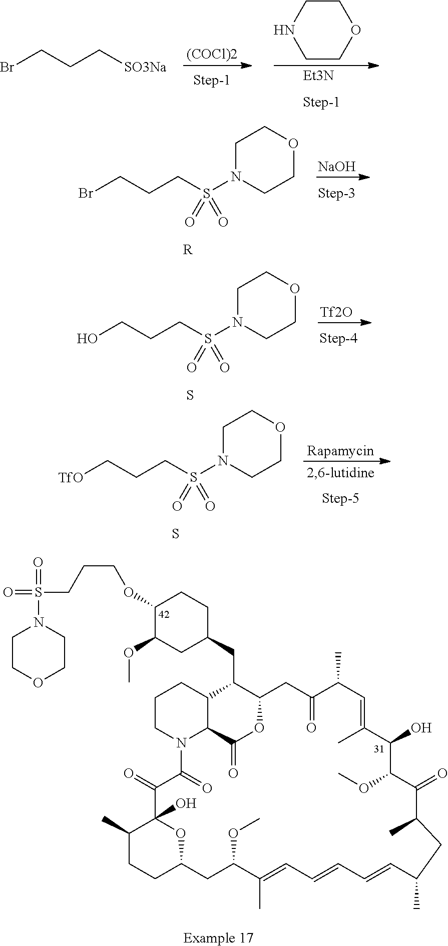

### Step-1 to Step-3: 3-(Morpholinosulfonyl)propyl trifluoromethanesulfonate (S)

To a suspension of sodium 3-bromopropane-1-sulfonate (2.26 g, 10 mmol) and DMF (4 drops) in THF—CH₂Cl₂ (1:1, 20 mL) was slowly added oxalyl chloride (2 mL, 15 mmol) at 0° C. After addition, the reaction mixture was filtered. The filtrate was concentrated to give crude bromoalkylsulfonyl chloride which was dissolved in CH₂Cl₂ (20 mL). To this reaction solution was added the solution of morpholine (1.5 mL, 24 mmol) and triethylamine (3 mL, 40 mmol) in CH₂Cl₂ (10 mL) at 0° C. The reaction mixture was stirred for 3 h and filtered. The filtrate was washed with dilute hydrochloride acid twice, dried over Na₂SO₄, and concentrated to afford crude 4-(3-bromopropylsulfonyl)morpholine (R) which was dissolved in THF (10 mL). Aqueous NaOH (0.12 g, 30 mL) solution was added in the above solution. The mixture was stirred for 5 h at 90° C., cooled to room temperature, and extracted with ethyl acetate. The organic layer was washed with water, dried over anhydrous Na₂SO₄, and concentrated. The residue was purified by silica gel chromatography eluted with ethyl acetate-petroleum ether (3:1) to obtain 4-(3-bromopropylsulfonyl)morpholine as white solid (S) (0.09 g, 30%).

### Step-4 and Step-5: 42-O-[3-(Morpholinosulfonyl)propyl]-rapamycin

To the solution of 2,6-lutidine (1.4 mL, 12 mmol) and 4-(3-hydroxypropylsulfonyl)morpholine (1.28 g, 6 mmol) in anhydrous CH₂Cl₂ (20 mL) was added the solution of trifluoromethanesulfonic anhydride (1.1 mL, 6 mmol) in anhydrous CH₂Cl₂ (10 mL) dropwise at −78° C. under N₂ atmosphere. Upon addition, the reaction mixture was warmed to −20° C., stirred for 1 h, and cooled back to −78° C. To the above reaction system was added rapamycin (2.7 g, 3 mmol). The resultant mixture was warmed to room temperature, stirred overnight, diluted with Na₂CO₃ aqueous solution (10%, 30 mL), and exacted with CH₂Cl₂ (20 mL). The organic layer was washed with brine (20 mL), dried over anhydrous Na₂SO₄ and concentrated. The residue was purified by silica gel chromatography eluted with ethyl acetate-methanol (20:1) to afford the title compound as a white solid (0.21 g, 10%).

¹H NMR (400 MHz, CDCl₃) δ 3.27-3.28 (m, 5H), 3.72-3.78 (m, 10H), 4.76 (s, 1H), 5.15-5.50 (m, 4H), 5.94-6.38 (m, 4H).

m/z (relative intensity, %): 1106 [M+1]^{+.} (100).

The following examples were prepared according to the methods outlined herein:

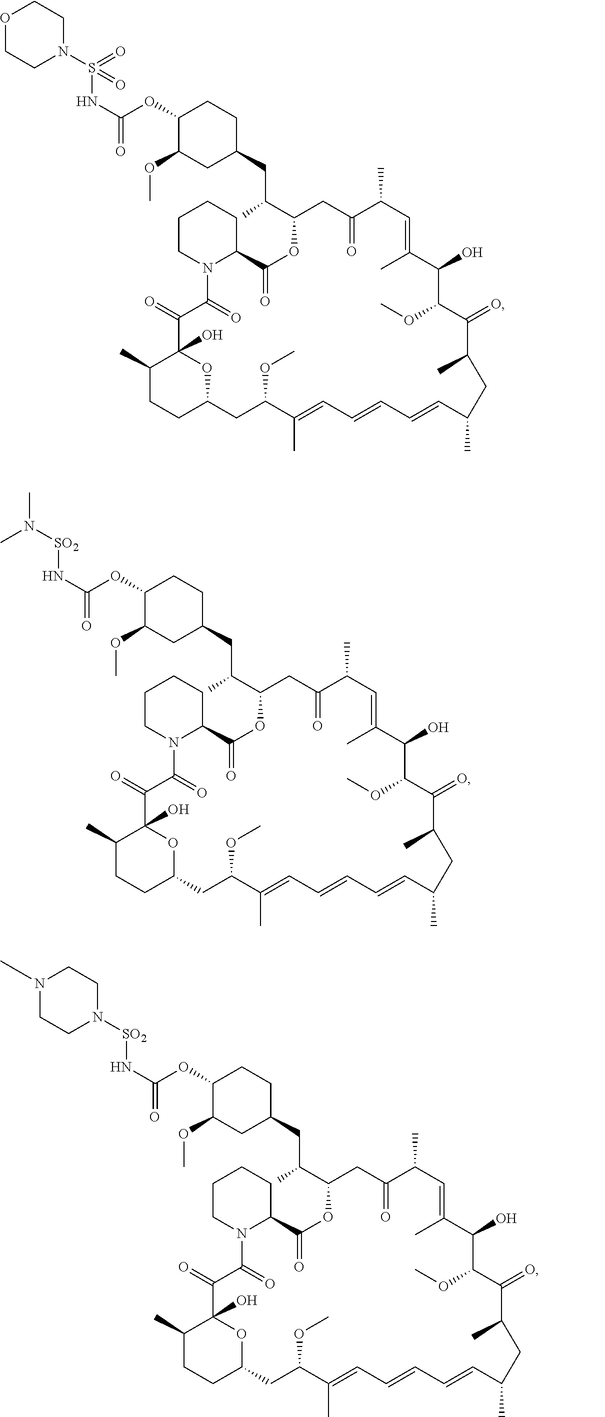

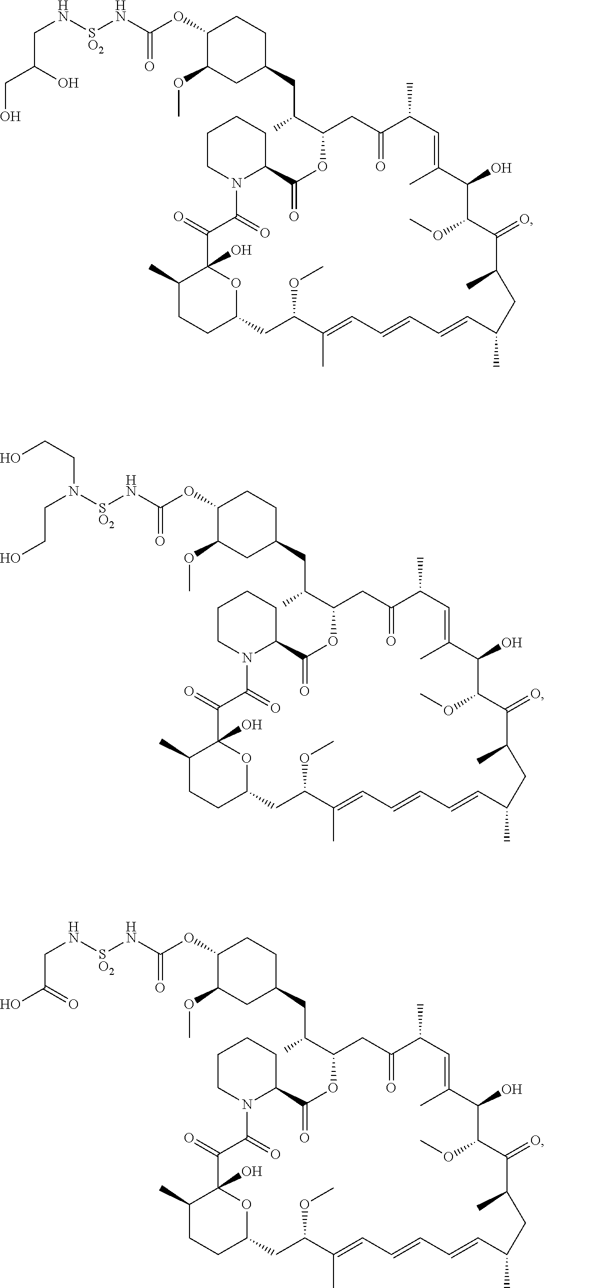

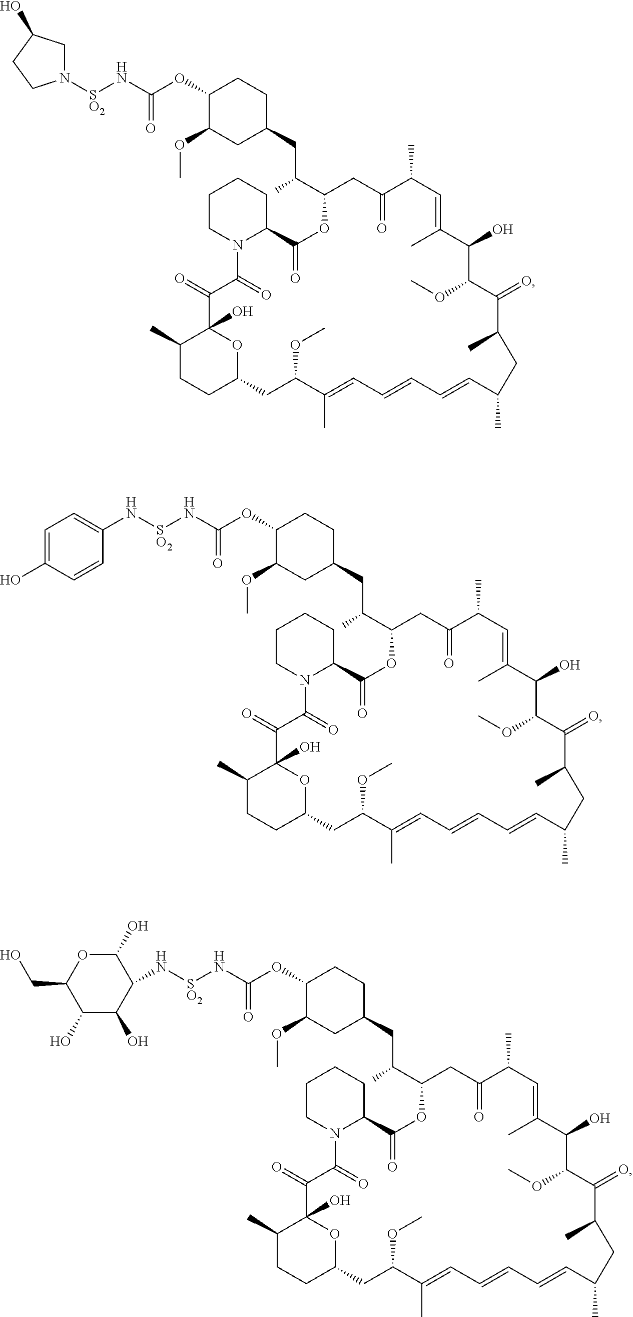

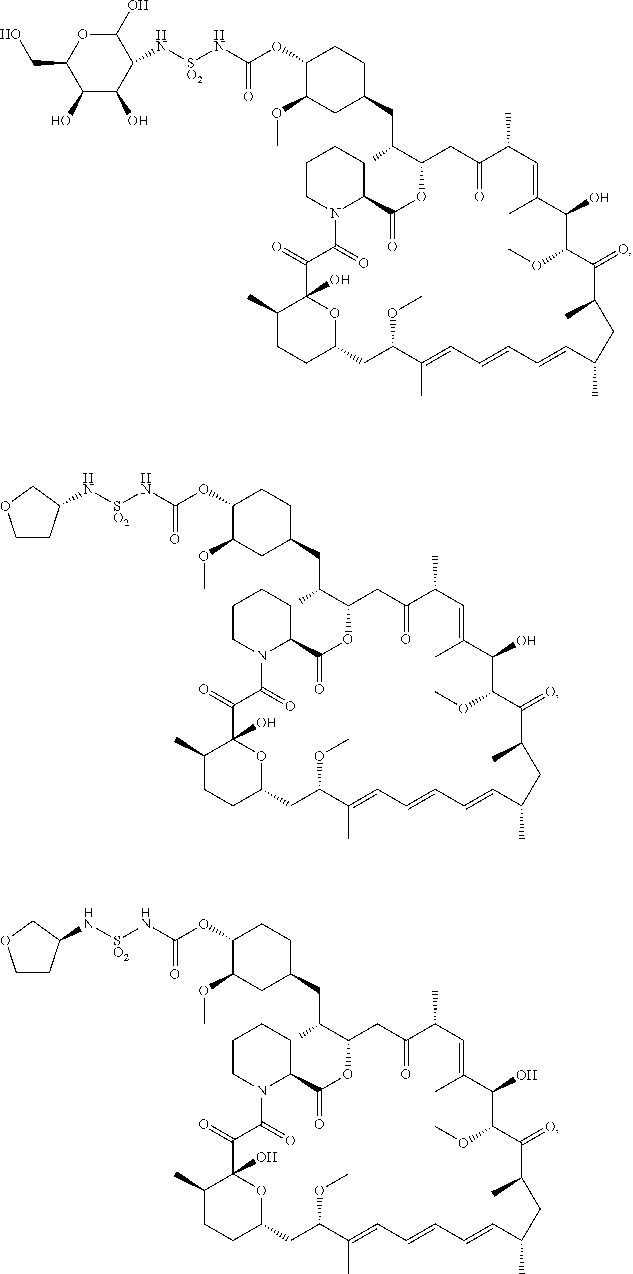

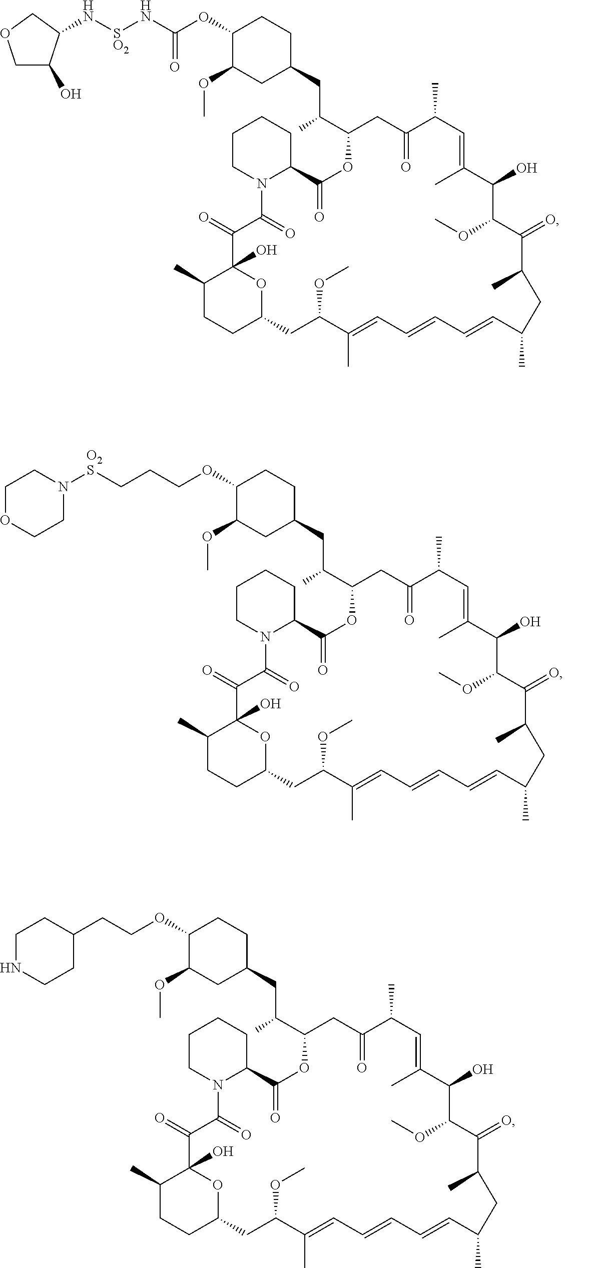

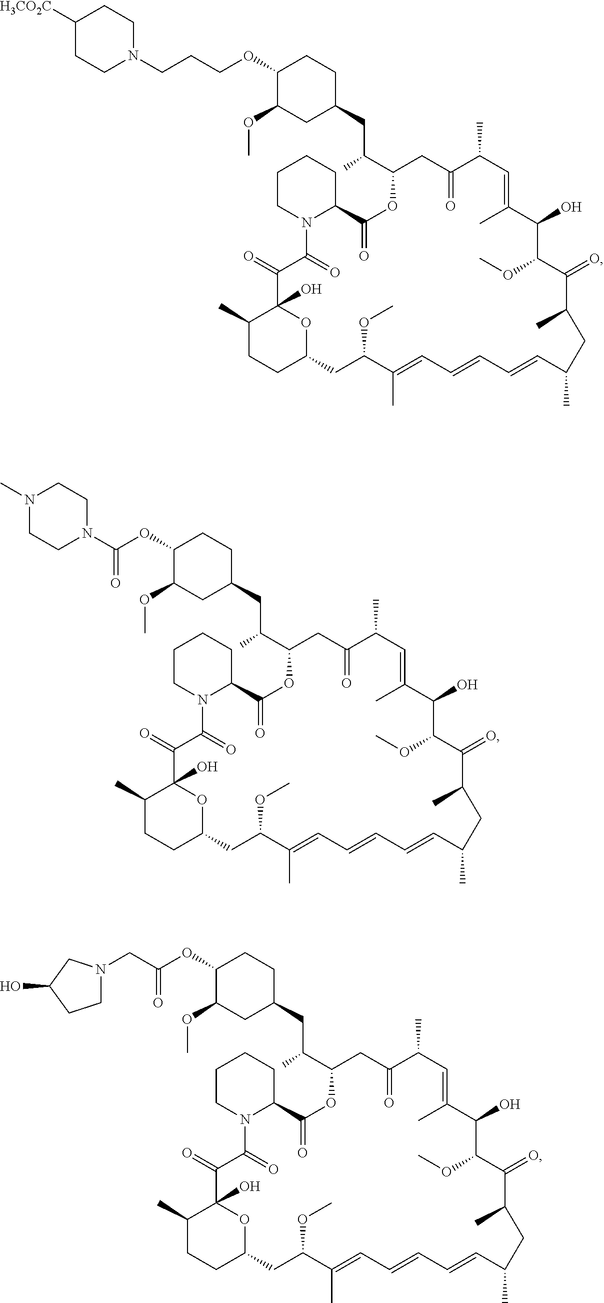

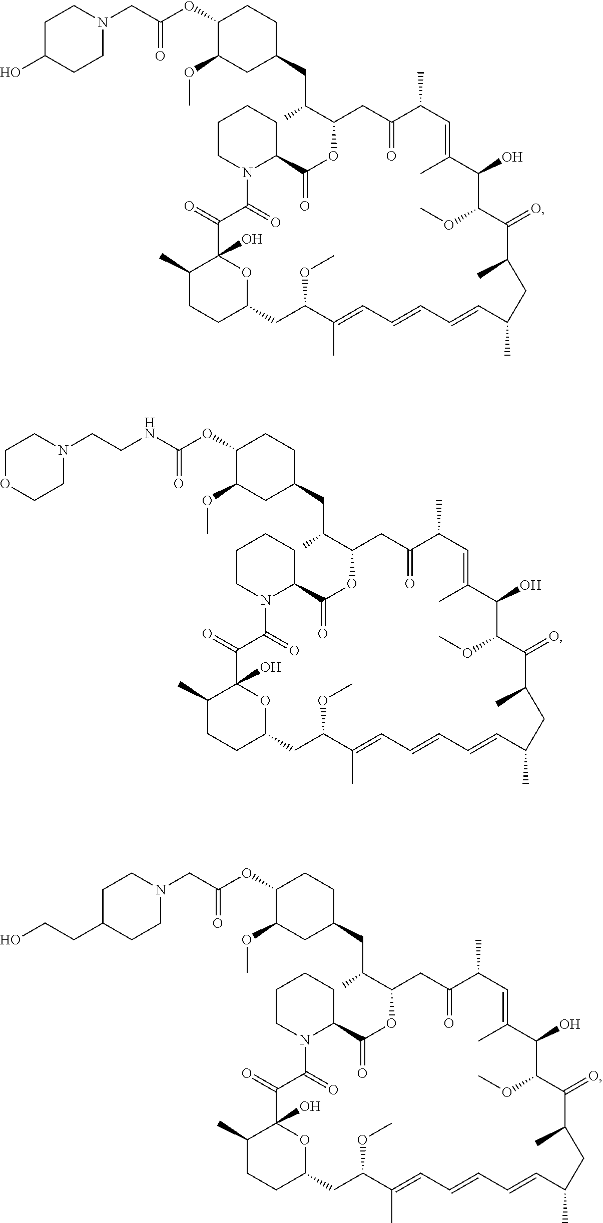

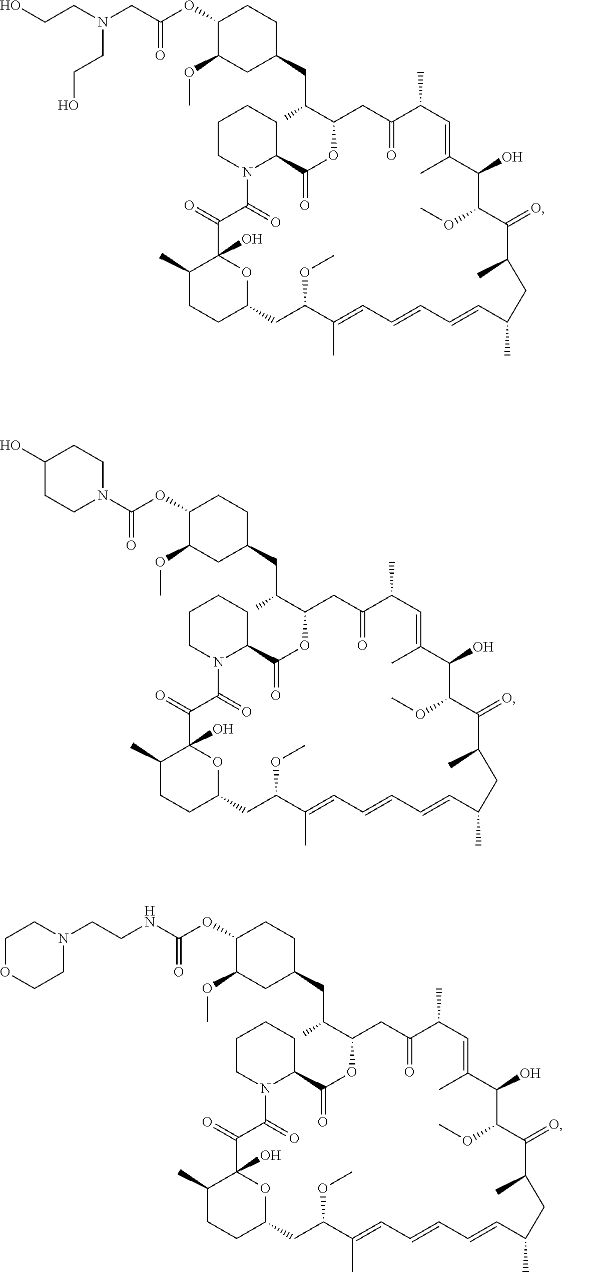

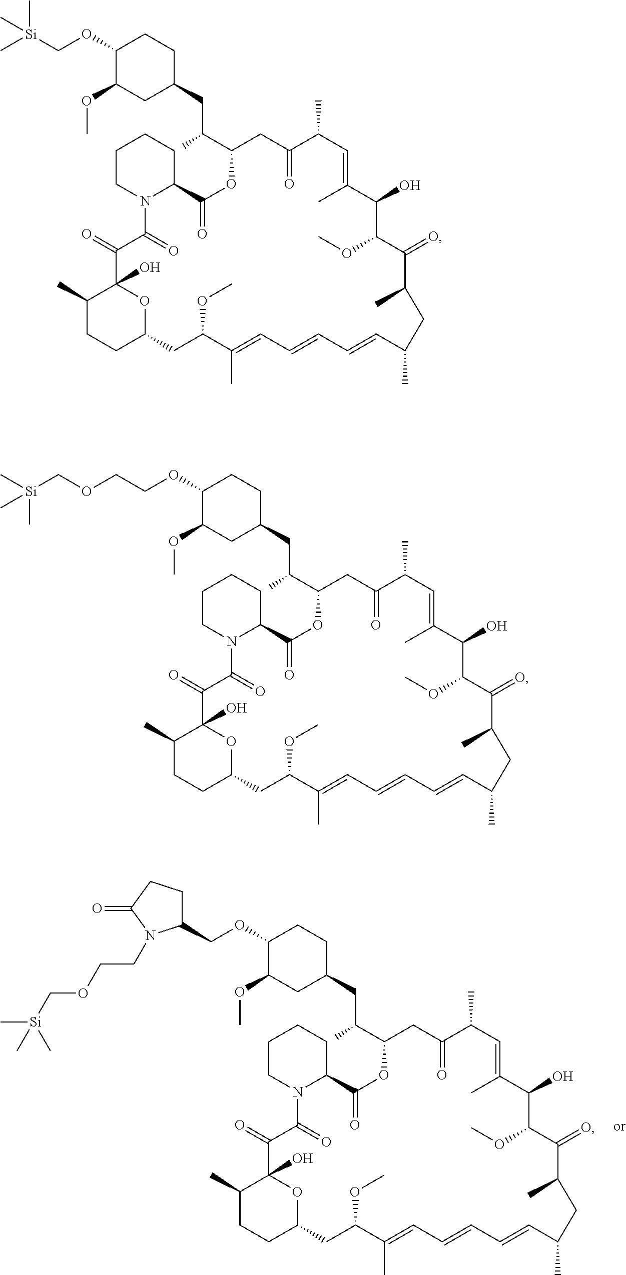

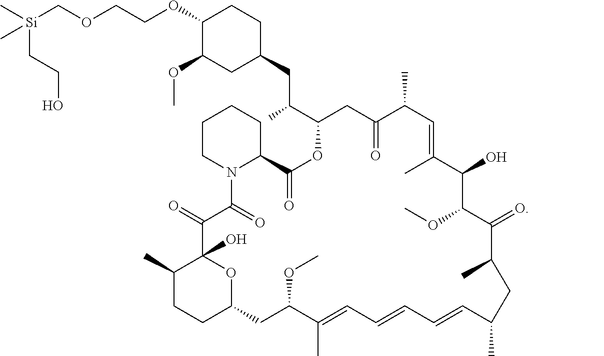

### Example 18

### mTOR Assay Screening Protocol

Kit: Calbiochem Cat. No. CBA055: K-LISA mTOR Activity Kit

### Preparation of Reagents

The following table provides reagent preparation instructions to obtain the volume of Working Solutions (WS) required for 10 wells. Volumes can be scaled to process more samples and provide overage for pipetting error.

Reagent Volume (per 10-well strip) Composition:
mTOR Substrate WS: 1 ml
Dilution factor: 1:400

### 997.5 μl TBS

2.5 μl mTOR Substrate

### 2× Kinase Assay Buffer WS: 500 μl

### 500 μl 2× Kinase Assay Buffer

5 μl ATP solution

### 10 μl 100 mM DTT

Anti p70S6K-T389 WS: 1 ml
Dilution factor: 1:1000

### 999 μl Antibody Diluent

### 1 μl Anti-p70S6K-T389

### HRP-Antibody Conjugate WS: 1 ml

Dilution factor: 1:400

### 997.5 μl Antibody Diluent

### 2.5 μl HRP-Antibody Conjugate

### Plate Wash (1×): 20 ml

Dilution factor: 1:10
9 ml dH2O

### 1 ml 10× Plate Wash Concentrate

FKBP12: Sigma F5398 FK-Binding Protein human, recombinant, expressed in Escherichia coli

### Detailed Protocol

A. Protocol for mTOR Kinase Activity and Inhibitor Screening:
1. Remove the required number of strips from the Glutathione-Coated 96-Well Plate and place them in the 96-well frame.
2. Add 100 μl mTOR Substrate WS to each well and incubate for 1 h at room temperature.
3. When performing inhibitor screening/testing, pre-incubate 50 μl mTOR Standard (from step 2) with test inhibitor or wortmannin in a separate tube on ice for 20 min (for example 50 μl mTOR Standard with 1 μl Woltmannin, 50×). This pre-incubation can be carried out during the incubation in step 2.
For inhibitor testing: weigh out inhibitor and resuspend in DMSO to a concentration of 5 mM prior to addition to assay
Test concentrations for inhibitors in assay ranged from 10 uM-0.0048 uM (final concentrations)
Inhibitor reaction contained: mTOR Standard, inhibitor (at indicated concentration) and FKBP12 (at 35-37 ug/ml)
Dilutions of inhibitors made in TBS
Controls for assay include: mTOR Standard only, mTOR Substrate only (blank), FKBP12+mTOR Standard (no inhibitor), inhibitor compound+mTOR Standard (no FKBP12),
   4. Aspirate contents of the wells and wash each well of the Glutathione-Coated 96-Well Plate with 200 μl TBS. Empty the contents of the wells into the sink and dry the wells by tapping the inverted plate on paper towels. Repeat for a total of 3 washes.
   5. Add the following components to each well in the specified order:

mTOR Standard or mTOR Sample* (diluted to assay range with phosphate-free buffer or water): 50 μl

### 2× Kinase Assay Buffer WS: 50 μl

### Total=100 μl

*Pre-incubated with inhibitor in step 3 or without inhibitor
6. Cover the plate with the Plate Sealer, mix with a plate shaker or equivalent for 30 seconds, and incubate for 30 minutes at 30° C.
7. Stop the kinase reaction by adding 10 μl Kinase Stop Solution to each well. (Reaction may also be stopped simply by discarding the contents of the wells).
8. Aspirate the contents of each well and wash with 200 μl Plate Wash (1×). Gently agitate the plate using a plate shaker or equivalent for 5 min. Empty the contents of the wells into the sink and dry the wells by tapping the inverted plate on paper towels. Wash the plate 2 more times without shaking the plate.
9. Add 100 μl Anti-p70S6K-T389 WS to each well, cover the plate with the Plate Sealer, and incubate 1 h at room temperature.
10. Wash the plate 4 times as outlined in step 8 without shaking the plate.
11. Add 100 μl HRP-Antibody Conjugate WS to each well, cover the plate with the Plate Sealer, and incubate 1 h at room temperature.
12. Wash the plate as outlined in step 10.
13. Add 100 μl TMB Substrate to each well, cover the plate with the Plate Sealer, and incubate 5-20 min at room temperature.
14. Add 100 pd ELISA Stop Solution to each well and read the absorbance at 450 nm, preferably with a reference wavelength set at 595 nm.
15. Analyze data using Microsoft Excel and GraphPad Prism software.

All exemplary compounds as shown below were found to have IC₅₀ values of less than 5 μM, many having IC₅₀ values of less than 1 μM, some of less than 0.1 μM.

TABLE 1
IC50 values of exemplary compounds

| Examples | IC50, μM |
|---|---|
| Example 1 | 0.01 |
| Example 2 | 0.03 |
| Example 3 | 0.11 |
| Example 4 | 0.01 |
| Example 5 | 0.001 |
| Example 6 | 2.5 |
| Example 7 | 0.17 |
| Example 8 | 0.07 |
| Example 11 | 2.3 |
| Example 12 | 0.03 |
| Example 13 | 0.07 |
| Example 14 | 0.24 |
| Example 15 | 0.21 |
| Example 16 | 0.3 |
| Example 17 | 0.14 |

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A compound of formula (I): 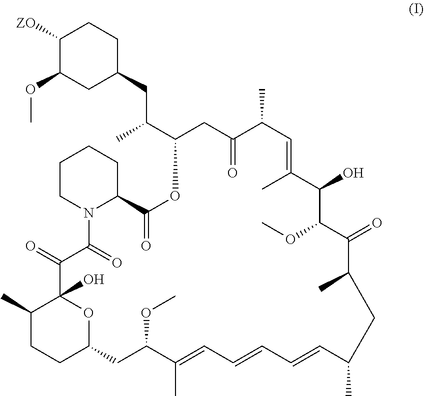
wherein Z comprises
(a) —C(O)NHS(O)₂N(R¹)(R²), wherein R¹ and R² are each independently H, alkyl, hydroxyalkyl, aminoalkyl, cycloalkyl, heterocyclyl, aryl, or heteroaryl, wherein any heterocyclyl independently comprises 1-3 heteroatoms comprising O, S, S(O), S(O)₂, or N; or, R¹ and R² together with a nitrogen atom to which they are bonded form a heterocycle ring which contains 0-3 additional heteroatoms comprising O, S, S(O), S(O)₂, or N, wherein any alkyl, hydroxyalkyl, aminoalkyl, aryl, heteroaryl, heterocyclyl, or heterocycle ring formed by R¹ and R² together with a nitrogen atom is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof;
R³ independently at each occurrence is H, alkyl, cycloalkyl, heterocyclyl, aryl, or heteroaryl, wherein any R³ except hydrogen is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and
R⁴ and R⁵ independently at each occurrence is H, alkyl, hydroxyalkyl, aminoalkyl, cycloalkyl, heterocyclyl, aryl, or heteroaryl; or R⁴ and R⁵ together with a nitrogen atom to which they are bonded comprises a saturated 5-, 6-, or 7-membered heterocyclic ring optionally comprising 0-3 additional heteroatoms comprising N, S S(O), S(O)₂ or O; wherein any alkyl, hydroxyalkyl, aminoalkyl, cycloalkyl, heterocyclyl, aryl, or heteroaryl, or heterocyclic ring formed by R⁴ and R⁵ together with a nitrogen atom, is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof;
or
(b) A-W—CH₂—(CH₂—O)ₘ—(CH(R))ₙ—, wherein
R is independently at each occurrence H or OR³;
m and n are each independently 0 to about 4;
W is a bond, C(O), C(O)C(O), S(O), S(O)₂, P(O)OR³, or P(O)NR⁴R⁵;
A comprises a saturated or unsaturated 5-, 6-, or 7-membered heterocycle containing one or more of N, O, S, S(O) or S(O)₂, wherein A is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and wherein A is bonded in any chemically feasible manner to CH₂;
R³ independently at each occurrence is H, alkyl, cycloalkyl, heterocyclyl, aryl, or heteroaryl, wherein any R³ except hydrogen is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and
R⁴ and R⁵ independently at each occurrence is H, alkyl, hydroxyalkyl, aminoalkyl, cycloalkyl, heterocyclyl, aryl, or heteroaryl; or R₄ and R₅ together with a nitrogen atom to which they are bonded comprises a saturated 5-, 6- or 7-membered heterocyclic ring comprising one or more of heteroatoms comprising N, S S(O), S(O)₂ or O; wherein any alkyl, hydroxyalkyl, aminoalkyl, alkylaminoalkyl, or aryl, or heterocyclic ring comprising R⁴ and R⁵ together, is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof;
or
(c) X—(CH₂)ₘYC(═O)(CH₂)ₙ—, wherein
m and n are each independently 0 to about 2;
Y is NR¹⁴, O, S, or a bond;
X comprises OR¹¹, or NR¹⁴R¹⁵, wherein
R¹¹ is H, alkyl, cycloalkyl, heterocyclyl, aryl, or heteroaryl, wherein any R¹¹ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;
R¹³ independently at each occurrence is H, alkyl, cycloalkyl, heterocyclyl, aryl, or heteroaryl, wherein any R¹³ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³OP(═O)(OR¹³)(OR¹³), OP(═O)(OR¹³)NR¹⁴R¹⁵, or P(OR¹³)(NR¹⁴R¹⁵), or, when pluri-substituted, any combination thereof;
R¹⁴ and R¹⁵ independently at each occurrence is H, alkyl, hydroxyalkyl, aminoalkyl, cycloalkyl, heterocyclyl, aryl, or heteroaryl; or R¹⁴ and R¹⁵ together with a nitrogen atom to which they are bonded comprises a saturated 5-, 6- or 7-membered heterocyclic ring optionally comprising one or more additional heteroatoms comprising N, S S(O), S(O)₂ or O; wherein any alkyl, hydroxyalkyl, aminoalkyl, alkylaminoalkyl, or aryl, or heterocyclic ring comprising R¹⁴ and R¹⁵ together with a nitrogen atom is optionally independently mono- or pluri-substituted with alkyl, hydroxyalkyl, aminoalkyl, halogen, oxo, OR¹³, NHCONR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵, or, when pluri-substituted, any combination thereof;
or
(d) —X-A¹-(CH₂)ₙ—Y¹—Si(R²²)(R²³)(R²⁴)
wherein X comprises ((CHR²¹)ₘ;
R²¹ is independently at each occurrence H, alkyl, hydroxyl, alkoxyl, or amino;
m and n are independently 0 to about 3;
Y¹ is a bond, O(CH₂)ᵣ, NR¹⁴(CH₂)ᵣ, or S(CH₂)ᵣ, wherein r is 0 to about 3;
A¹ is a bond, O, NR¹⁴, S, cycloalkyl, or heterocyclyl;
R²², R²³ and R²⁴ are each independently alkyl, hydroxyalkyl, aminoalkyl, alkoxy, cycloalkyl, heterocyclyl, aryl, or heteroaryl;
wherein any alkyl, cycloalkyl, heterocyclyl, alkoxy, aryl, or heteroaryl is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;
R¹³ independently at each occurrence is H, alkyl, cycloalkyl, heterocyclyl, aryl, or heteroaryl, wherein any R¹³ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³OP(═O)(OR¹³)(OR¹³), OP(═O)(OR¹³)NR¹⁴R¹⁵, or P(OR¹³)(NR¹⁴R¹⁵), or, when pluri-substituted, any combination thereof;
R¹⁴ and R¹⁵ independently at each occurrence is H, alkyl, hydroxyalkyl, aminoalkyl, cycloalkyl, heterocyclyl, aryl, or heteroaryl; or R¹⁴ and R¹⁵ together with a nitrogen atom to which they are bonded comprises a saturated 5-, 6- or 7-membered heterocyclic ring optionally comprising one or more additional heteroatoms comprising N, S S(O), S(O)₂ or O; wherein any alkyl, hydroxyalkyl, aminoalkyl, alkylaminoalkyl, or aryl, or heterocyclic ring comprising R¹⁴ and R¹⁵ together with a nitrogen atom is optionally independently mono- or pluri-substituted with alkyl, hydroxyalkyl, aminoalkyl, halogen, oxo, OR¹³, NHCONR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³ OP(═O)(OR¹³)(OR¹³), OP(═O)(OR¹³)NR¹⁴R¹⁵, or P(OR¹³)(NR¹⁴R¹⁵) or, when pluri-substituted, any combination thereof.

2. The compound of claim 1 wherein Z comprises —C(O)NHS(O)₂N(R¹)(R²), wherein R¹ and R² are each independently H, alkyl, hydroxyalkyl, aminoalkyl, cycloalkyl, heterocyclyl, aryl, or heteroaryl, wherein any heterocyclyl independently comprises 1-3 heteroatoms comprising O, S, S(O), S(O)₂, or N; or, R¹ and R² together with a nitrogen atom to which they are bonded form a heterocycle ring which contains 0-3 additional heteroatoms comprising O, S, S(O), S(O)₂, or N, wherein any alkyl, hydroxyalkyl, aminoalkyl, aryl, heteroaryl, heterocyclyl, or heterocycle ring formed by R¹ and R² together with a nitrogen atom is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof;
R³ independently at each occurrence is H, alkyl, cycloalkyl, heterocyclyl, aryl, or heteroaryl, wherein any R³ except hydrogen is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and
R⁴ and R⁵ independently at each occurrence is H, alkyl, hydroxyalkyl, aminoalkyl, cycloalkyl, heterocyclyl, aryl, or heteroaryl; or R⁴ and R⁵ together with a nitrogen atom to which they are bonded comprises a saturated 5-, 6-, or 7-membered heterocyclic ring optionally comprising 0-3 additional heteroatoms comprising N, S S(O), S(O)₂ or O; wherein any alkyl, hydroxyalkyl, aminoalkyl, cycloalkyl, heterocyclyl, aryl, or heteroaryl, or heterocyclic ring formed by R⁴ and R⁵ together with a nitrogen atom, is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof.

3. The compound of claim 2, wherein R¹ and R² together with the nitrogen atom to which they are bonded comprises a heterocyclyl.

4. The compound of claim 2 wherein R¹ is H.

5. The compound of claim 4 wherein R² is 2,3-dihydroxylpropyl, carboxymethyl, substituted or unsubstituted phenyl, glycosyl, or a substituted or unsubstituted tetrahydrofuranyl group.

6. The compound of claim 2 wherein R¹ and R² are each independently methyl or hydroxyethyl.

7. The compound of claim 2, wherein the compound of formula (I) comprises 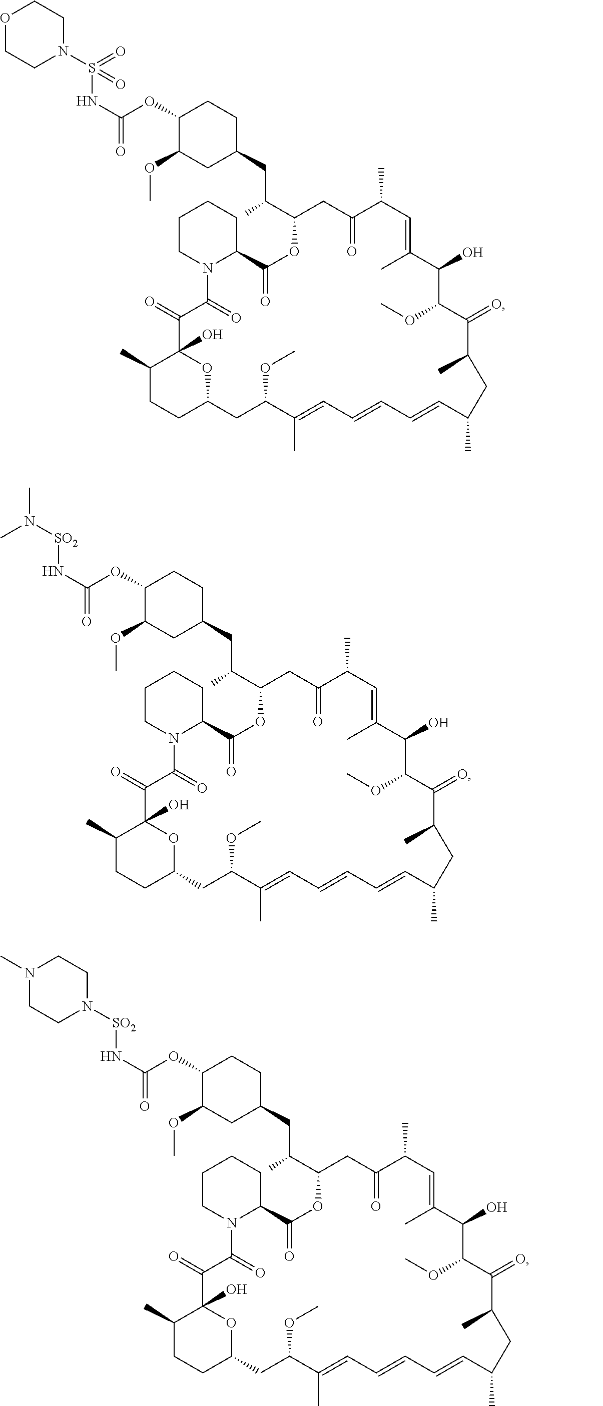 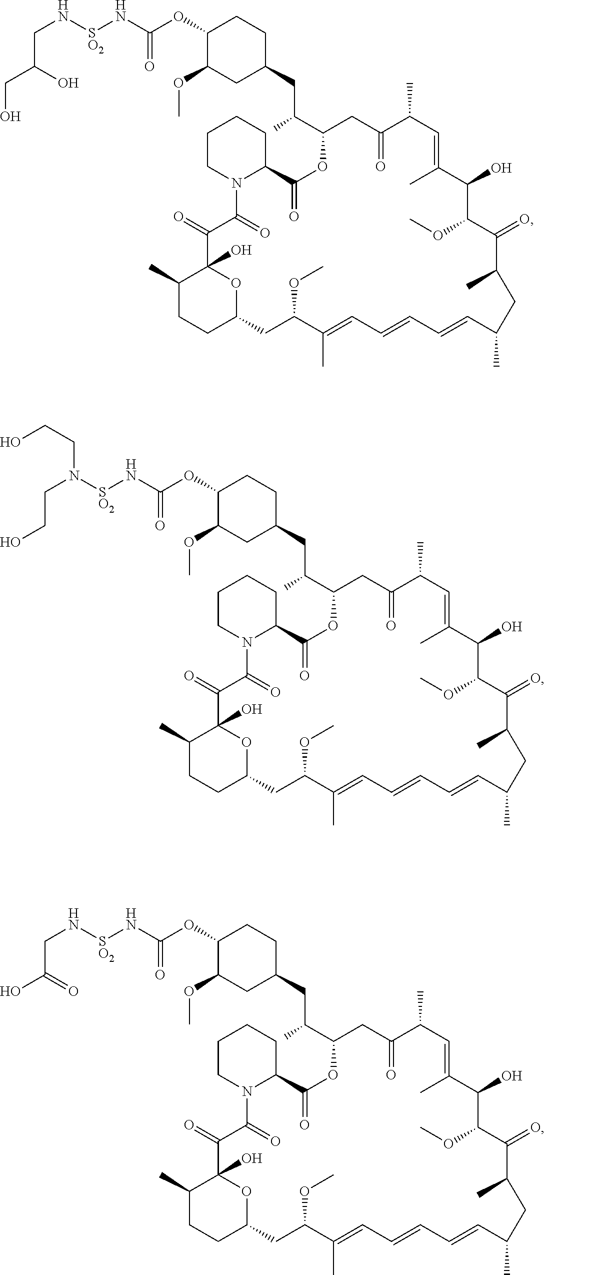 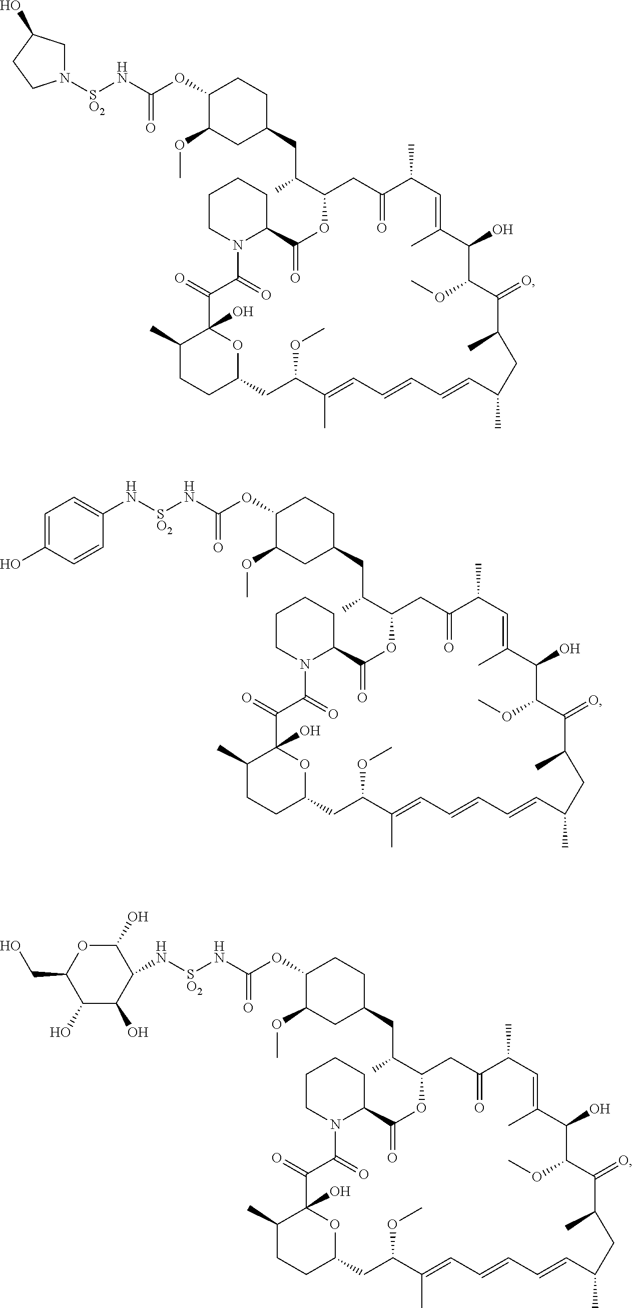 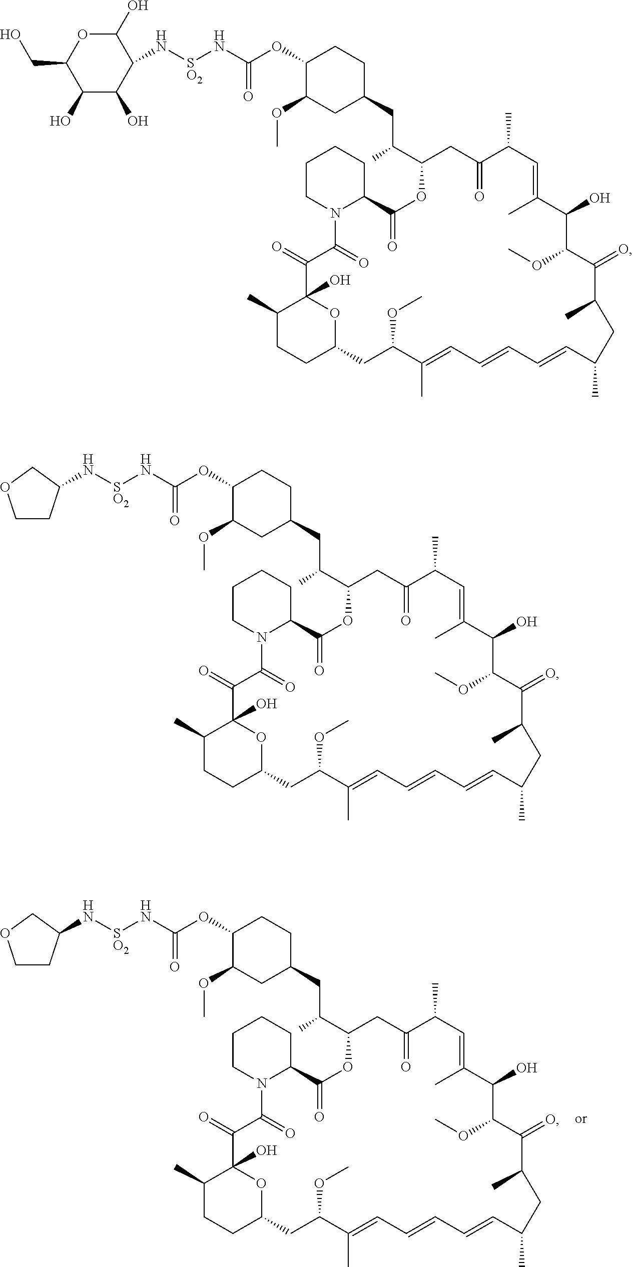 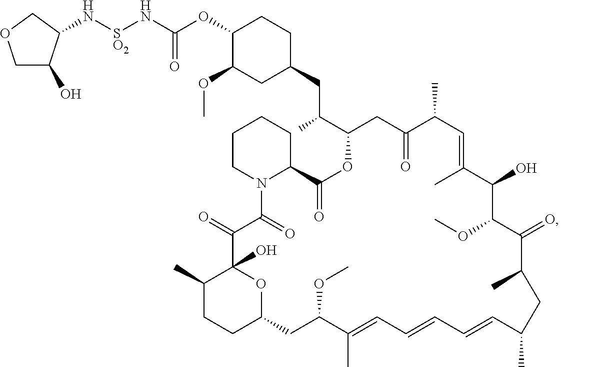
or any pharmaceutically acceptable salt thereof.

8. The compound of claim 1 wherein Z comprises A-W—CH₂—(CH₂—O)ₘ—(CH(R))ₙ—, wherein
R is independently at each occurrence H or OR³;
m and n are each independently 0 to about 4;
W is a bond, C(O), C(O)C(O), S(O), S(O)₂, P(O)OR³, or P(O)NR⁴R⁵;
A comprises a saturated or unsaturated 5-, 6-, or 7-membered heterocyclyl containing one or more of N, O, S, S(O) or S(O)₂, wherein A is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and wherein A is bonded in any chemically feasible manner to CH₂;
R³ independently at each occurrence is H, alkyl, cycloalkyl, heterocyclyl, aryl, or heteroaryl, wherein any R³ except hydrogen is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof; and
R⁴ and R⁵ independently at each occurrence is H, alkyl, hydroxyalkyl, aminoalkyl, cycloalkyl, heterocyclyl, aryl, or heteroaryl; or R⁴ and R⁵ together with a nitrogen atom to which they are bonded comprises a saturated 5-, 6- or 7-membered heterocyclic ring comprising one or more of heteroatoms comprising N, S S(O), S(O)₂ or O; wherein any alkyl, hydroxyalkyl, aminoalkyl, alkylaminoalkyl, or aryl, or heterocyclic ring comprising R⁴ and R⁵ together, is optionally independently mono- or pluri-substituted with alkyl, hydroxyalkyl, aminoalkyl, halogen, oxo, OR³, NHCONR⁴R⁵, NR⁴R⁵, COR³, COOR³, OC(O)R³, CONR⁴R⁵, OC(O)NR⁴R⁵, N(R⁴)C(O)R³, S(O)₂R³, S(O)₂OR³, OP(═O)(OR³)(OR³), OP(═O)(OR³)NR⁴R⁵, or P(OR³)(NR⁴R⁵), or, when pluri-substituted, any combination thereof.

9. The compound of claim 8 wherein R is H and, m is 0, and n is 1 or 2.

10. The compound of claim 8 wherein A is a bond or is SO₂.

11. The compound of claim 8 wherein W is piperidinyl or morpholinyl.

12. The compound of claim 8, wherein in the compound of formula (I) comprises 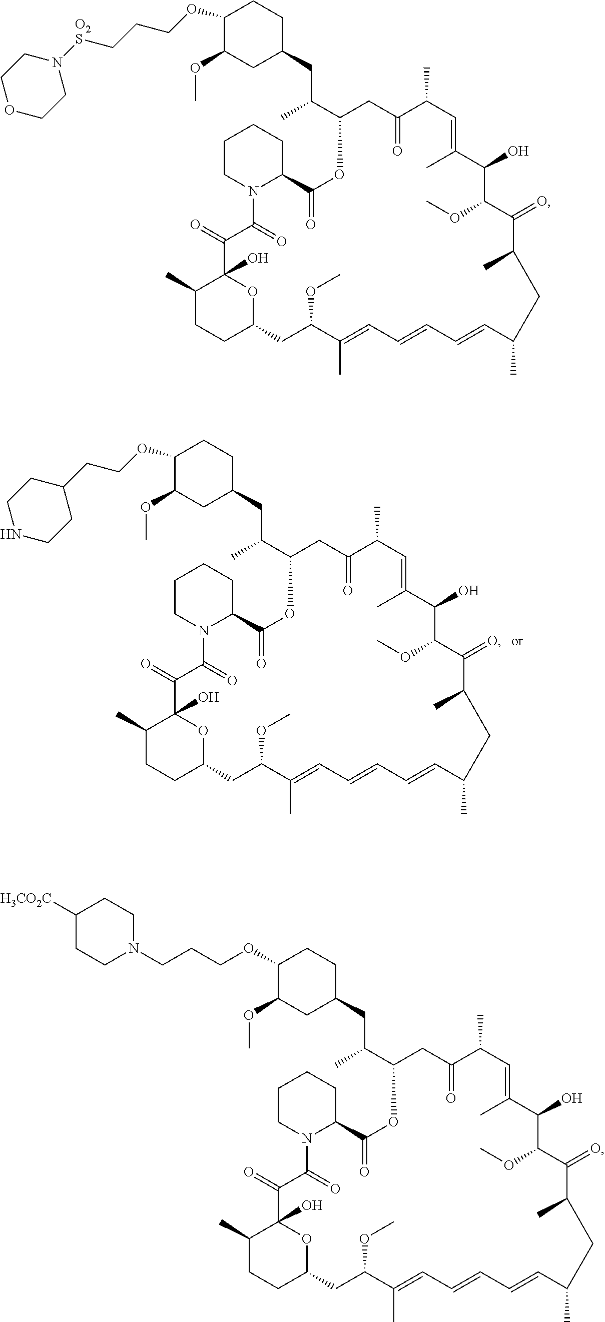
or any pharmaceutically acceptable salt thereof.

13. The compound of claim 1, wherein Z comprises X—(CH₂)ₘYC(═O)(CH₂)ₙ—, wherein
m and n are each independently 0 to about 2;
Y is NR¹⁴, O, S, or a bond;
X comprises OR¹¹, or NR¹⁴R¹⁵, wherein
R¹¹ is H, alkyl, cycloalkyl, heterocyclyl, aryl, or heteroaryl, wherein any R¹¹ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;
R¹³ independently at each occurrence is H, alkyl, cycloalkyl, heterocyclyl, aryl, or heteroaryl, wherein any R¹³ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴, R¹⁵, OC(O)NR¹⁴R¹⁵, N(R¹⁴)C(O)R¹³, S(O)₂R¹³, S(O)₂OR¹³OP(═O)(OR¹³)(OR¹³), OP(═O)(OR¹³)NR¹⁴R¹⁵, or P(OR¹³)(NR¹⁴R¹⁵), or, when pluri-substituted, any combination thereof;
R¹⁴ and R¹⁵ independently at each occurrence is H, alkyl, hydroxyalkyl, aminoalkyl, cycloalkyl, heterocyclyl, aryl, or heteroaryl; or R¹⁴ and R¹⁵ together with a nitrogen atom to which they are bonded comprises a saturated 5-, 6- or 7-membered heterocyclic ring optionally comprising one or more additional heteroatoms comprising N, S S(O), S(O)₂ or O; wherein any alkyl, hydroxyalkyl, aminoalkyl, alkylaminoalkyl, or aryl, or heterocyclic ring comprising R¹⁴ and R¹⁵ together with a nitrogen atom is optionally independently mono- or pluri-substituted with alkyl, hydroxyalkyl, aminoalkyl, halogen, oxo, OR¹³, NHCONR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³S(O)₂OR¹³, OP(═O)(OR¹³)(OR¹³), OP(═O)(OR¹³)NR¹⁴R¹⁵, or P(OR¹³)(NR¹⁴R¹⁵) or, when pluri-substituted, any combination thereof.

14. The compound of claim 13 wherein n is 0.

15. The compound of claim 13 wherein Y is NH or absent.

16. The compound of claim 13 wherein m is 1 or 2.

17. The compound of claim 13 wherein X comprises NR¹⁴R¹⁵.

18. The compound of claim 17 wherein NR¹⁴R¹⁵ comprises a substituted or unsubstituted heterocyclic ring, or wherein R¹⁴ and R¹⁵, or both, are independently hydroxyethyl.

19. The compound of claim 13, wherein in the compound of formula (I) comprises 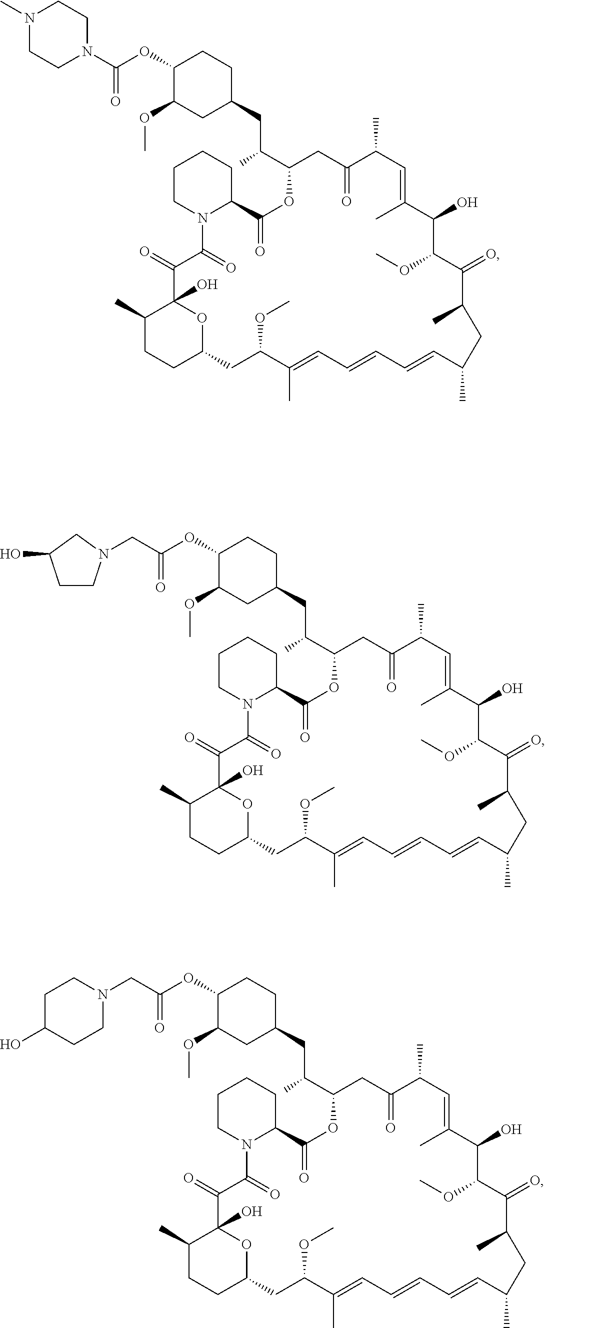 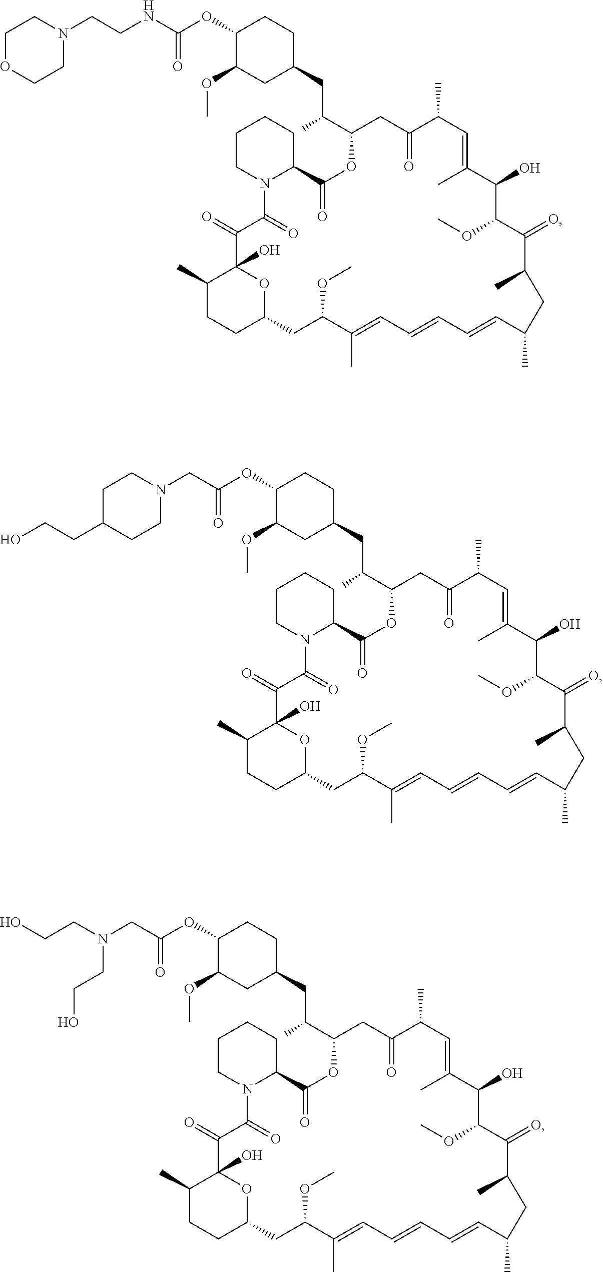 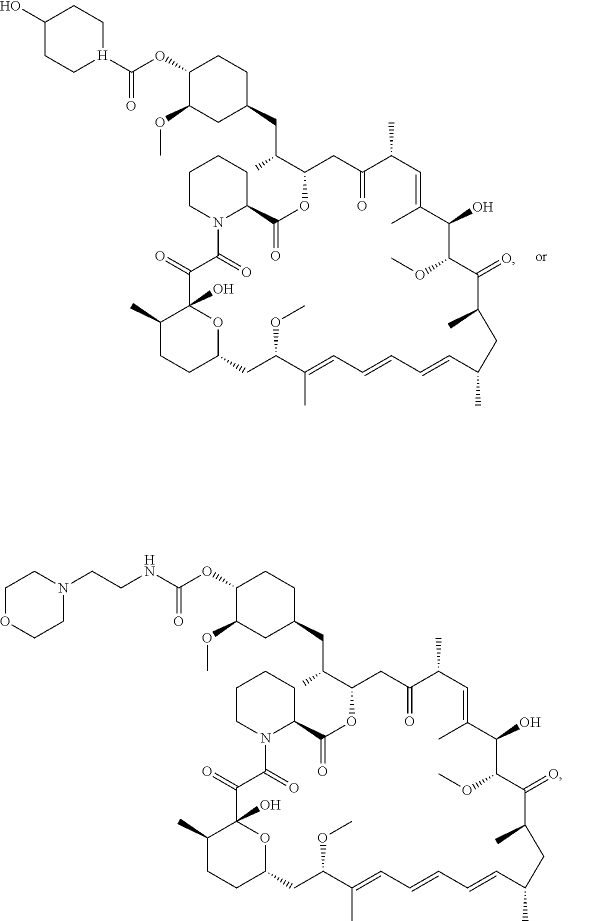
or any pharmaceutically acceptable salt thereof.

20. The compound of claim 1, wherein Z comprises —X-A¹-(CH₂)ₙ—Y¹—Si(R²²)(R²³)(R²⁴)
wherein X comprises ((CHR²¹)ₘ;
R²¹ is independently at each occurrence H, alkyl, hydroxyl, alkoxyl, or amino;
m and n are independently 0 to about 3;
Y¹ is a bond, O(CH₂)ᵣ, NR¹⁴(CH₂)ᵣ, or S(CH₂)ᵣ, wherein r is 0 to about 3;
A¹ is a bond, O, NR¹⁴, S, cycloalkyl, or heterocyclyl;
provided that when A and Y¹ are each a bond, and n is 0, a single bond exists between X and Si(R²²)(R²³)(R²⁴);
R²², R²³ and R²⁴ are each independently alkyl, hydroxyalkyl, aminoalkyl, alkoxy, cycloalkyl, heterocyclyl, aryl, or heteroaryl;
wherein any alkyl, cycloalkyl, heterocyclyl, alkoxy, aryl, or heteroaryl is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;
R¹³ independently at each occurrence is H, alkyl, cycloalkyl, heterocyclyl, aryl, or heteroaryl, wherein any R¹³ except hydrogen is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³, S(O)₂OR¹³, OP(═O)(OR¹³)(OR¹³), OP(═O)(OR¹³)NR¹⁴R¹⁵, or P(OR¹³)(NR¹⁴R¹⁵), or, when pluri-substituted, any combination thereof;
R¹⁴ and R¹⁵ independently at each occurrence is H, alkyl, hydroxyalkyl, aminoalkyl, cycloalkyl, heterocyclyl, aryl, or heteroaryl; or R¹⁴ and R¹⁵ together with a nitrogen atom to which they are bonded comprises a saturated 5-, 6- or 7-membered heterocyclic ring optionally comprising one or more additional heteroatoms comprising N, S S(O), S(O)₂ or O; wherein any alkyl, hydroxyalkyl, aminoalkyl, alkylaminoalkyl, or aryl, or heterocyclic ring comprising R¹⁴ and R¹⁵ together with a nitrogen atom is optionally independently mono- or pluri-substituted with alkyl, hydroxyalkyl, aminoalkyl, halogen, oxo, OR¹³, NHCONR¹⁴R¹⁵, NR¹⁴R¹⁵, COR¹³, COOR¹³, OC(O)R¹³, CONR¹⁴R¹⁵, OC(O)NR¹⁴R¹⁵, N(R¹⁴)C(O)R¹³, S(O)₂R¹³S(O)₂OR¹³, OP(═O)(OR¹³)(OR¹³), OP(═O)(OR¹³)NR¹⁴R¹⁵, or P(OR¹³)(NR¹⁴R¹⁵) or, when pluri-substituted, any combination thereof.

21. The compound of claim 20 wherein R²², R²³ and R²⁴ each independently is methyl or hydroxyethyl.

22. The compound of claim 20 wherein m is 0, A¹ is a bond, n is 2 and Y¹ is OCH₂.

23. The compound of claim 20 wherein A¹ is heterocyclyl.

24. The compound of claim 20, wherein in the compound of formula (I) comprises 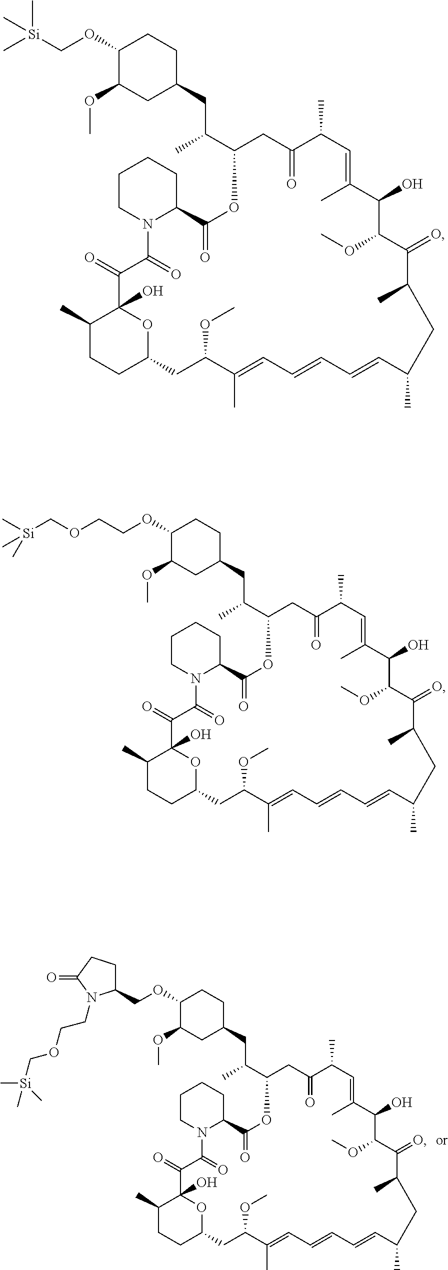 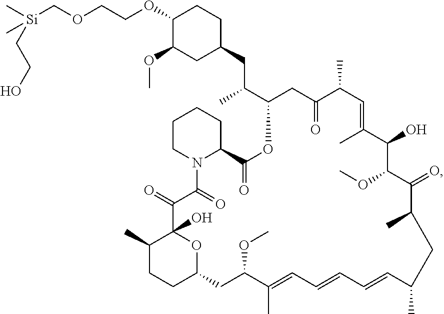
or any pharmaceutically acceptable salt thereof.

25. A pharmaceutical composition comprising an effective amount of a compound of claim 1 and a pharmaceutically acceptable excipient.

26. A pharmaceutical combination comprising an effective amount a compound of claim 1 and an effective amount of a second medicament.

27. A pharmaceutical composition comprising the combination of claim 26 and a pharmaceutically acceptable excipient.

28. A method of inhibiting the mTOR function of FKBP comprising contacting FKBP and an effective amount of the compound of claim 1.

29. A method of treating a malcondition wherein binding of a ligand to FKBP, or inhibition of the mTOR function of FKBP, or both, is medically indicated, comprising administering the compound of claim 1, to the patient in a dose, at a frequency of administration and for a duration of time sufficient to provide a beneficial effect to the patient.

30. The method of claim 29, wherein the malcondition comprises cancer.

31. The method of claim 30, wherein the cancer comprises a malignant solid tumor or a hematopoietic malignancy.

32. The method of claim 30, further comprising administering an effective amount of a known second anticancer medicament to the patient.

33. (canceled)

34. A method of preparation of a compound of claim 2, comprising contacting a compound of formula (II): 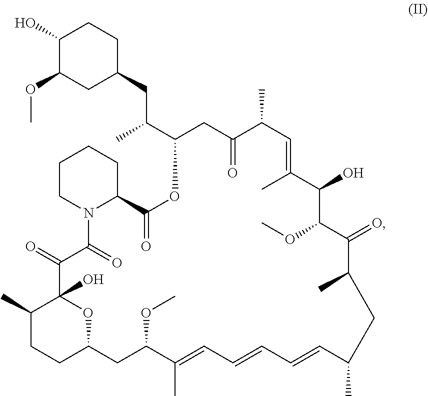
with a hydroxyl-protecting group reagent, to provide a compound of formula (III): 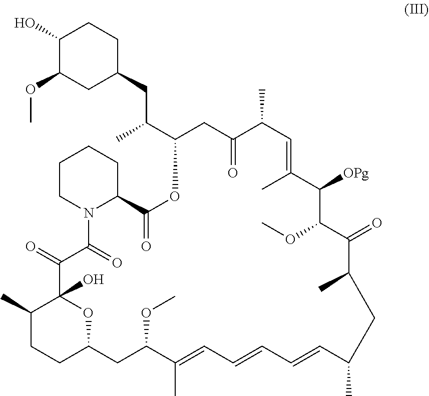
wherein Pg is a hydroxyl protecting group, then
contacting the compound of formula (III) with sulfurisocyanatidic chloride to provide a compound of formula (IV): 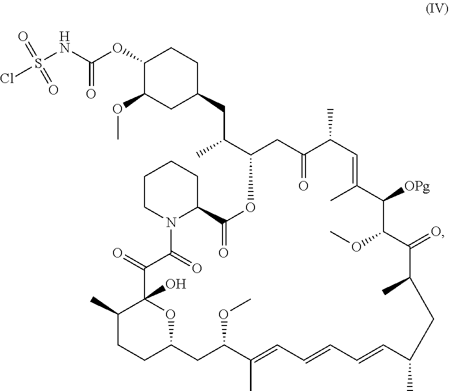
then, contacting the compound of formula (IV) with NH(R¹)(R²) to provide the compound of claim 2.

35. The method of claim 34 wherein the hydroxyl protecting group is a silyl ether.

36. A method of preparing a compound of claim 8 comprising contacting a compound of formula (II): 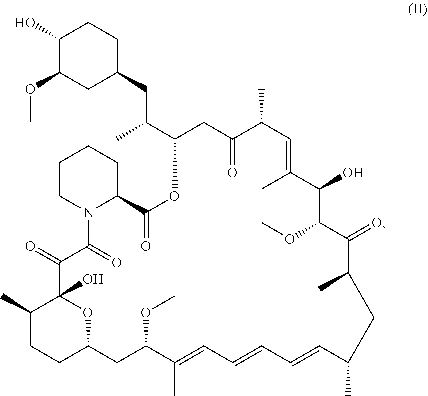
and a compound of formula Z-Lg, wherein Lg is a leaving group, to provide the compound of claim 8.

37. The method of claim 36 wherein the compound of formula Z—X is a compound of formula Z—O-Tf, wherein Tf signifies a triflate ester.

38. The method of claim 36 wherein the hydroxyl protecting group is a silyl ether.

39. A method of preparing a compound of claim 13 comprising contacting a compound of formula (II): 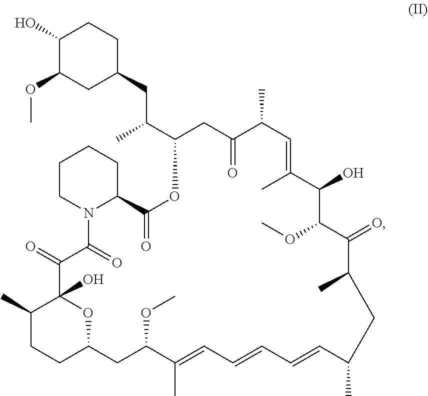
with a hydroxyl-protecting group reagent, to provide a compound of formula (III): 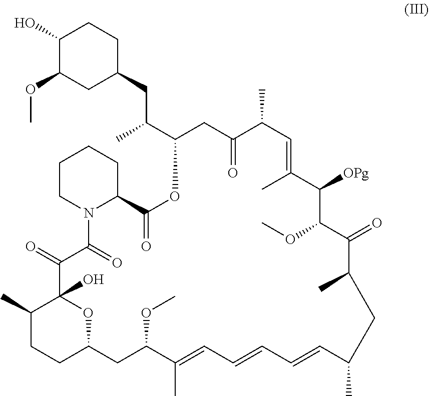
wherein Pg is a hydroxyl protecting group, then
contacting the compound of formula (III) with a reagent of formula Z-phenyl-O—C(═O)-Lg, wherein Z is one or more electron withdrawing groups disposed on phenyl and Lg is a leaving group, to provide a compound of formula (VI): 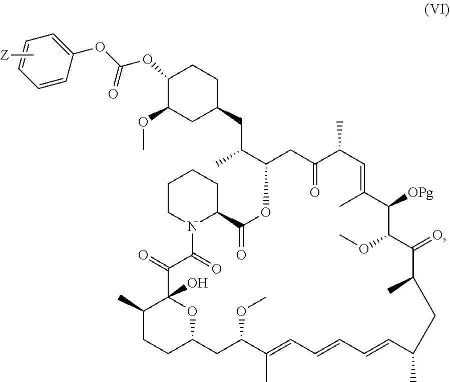
wherein Z signifies the one or more electron withdrawing groups; then, contacting the compound of formula (VI) with NH(R¹²)(R¹³) to provide the compound of claim 13.

40. The method of claim 39 wherein the reagent of formula Z-phenyl-O—C(═O)-Lg is a mononitrophenoxycarbonyl chloride or a dinitrophenoxycarbonyl chloride.

41. The method of claim 39 wherein the hydroxyl protecting group is a silyl ether.

42. A method of preparing a compound of claim 13, comprising contacting a compound of formula (II): 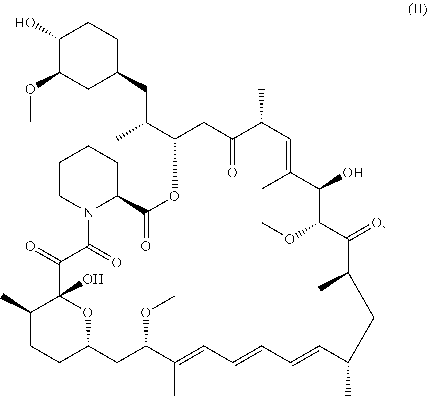
with a hydroxyl-protecting group reagent, to provide a compound of formula (III): 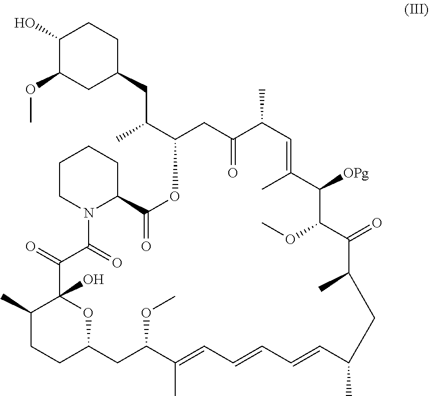
wherein Pg is a hydroxyl protecting group, then
contacting the compound of formula (III) with an activated haloacetate to provide a compound of formula (VII): 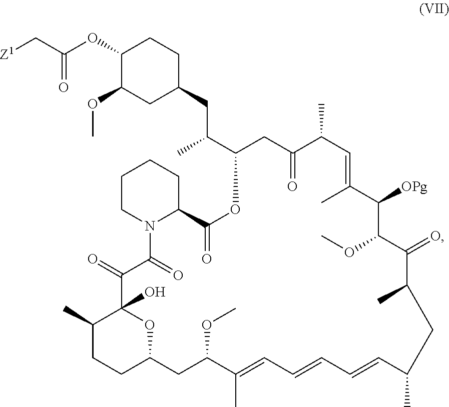
wherein Z¹ is a halogen;
then, contacting the compound of formula (VII) with NH(R¹²)(R¹³) to provide the compound of claim 13.

43. The method of claim 42 wherein Z¹ is bromo.

44. The method of claim 42 wherein the hydroxyl protecting group is a silyl ether.

45. A method of preparing a compound of claim 20, comprising contacting a compound of formula (II): 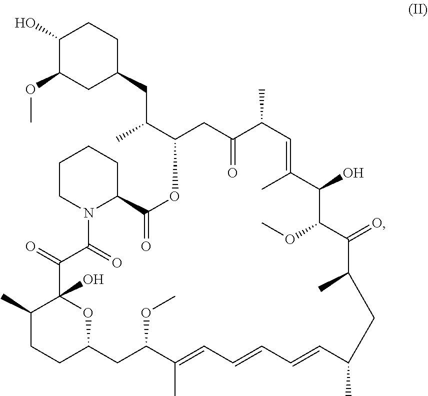
and a compound of formula Lg-A¹-(CH₂)ₙ—Si(R²²)(R²³)(R²⁴), wherein Lg is a leaving group, to provide the compound of claim 20.

46. The method of claim 45 wherein Lg is —O—SO₂CF₃.

47. The method of claim 45 wherein the hydroxyl protecting group is a silyl ether.
